(19) United States
(12) Patent Application Publication
(10) Pub. No.: US 2010/0068224 A1
(43) Pub. Date: Mar. 18, 2010

(54) Method for Producing Viral Vaccine and Therapeutic Peptide Antigens

(76) Inventors: Roberto CREA, San Mateo, CA (US); Randy Shen, Sunnyvale, CA (US)

Correspondence Address: King & Spalding LLP, P.O. Box 889, Belmont, CA, 94002-0889, US

(21) Appl. No.: 12/297,162

(22) PCT Filed: Apr. 24, 2007
(86) PCT No.: PCT/US07/10106

§ 371 Date: Dec. 1, 2009

Related U.S. Application Data
(60) Provisional application No. 60/794,880, filed on Apr. 24, 2006.

(51) Int. Cl. A61K 39/295; A61P 31/16

(52) U.S. Cl. 424/202.1

(57) ABSTRACT

A method of producing viral antigens for use in a vaccine or therapeutic composition against infection by a virus, and a composition produced thereby, are disclosed. The method is applicable to viruses having a plurality of subtypes whose protein sequences for at least one viral protein or polyprotein are known. Regions of a selected viral protein that (i) are known or predicted to bind to MHC proteins in host antigen-presenting cells, and (ii) have a moderate amino acid variability within the virus subtypes are systematically varied in amino acid sequence, and tested for enhanced binding to MHC proteins. The composition includes those peptides having the highest binding affinity for MHC proteins.

### FIELD OF THE INVENTION

The present invention relates to a method for producing novel peptides for use in viral vaccine and therapeutic compositions that are tailored for specific MHC presentation in target populations.

### BACKGROUND OF THE INVENTION

Host defense is a hallmark of vertebrate immune systems. To this end, antibodies perform numerous functions in the defense against pathogens. For instance, antibodies can neutralize a biologically active molecule, induce the complement pathway, stimulate phagocytosis (opsonization), or participate in antibody-dependent cell-mediated cytotoxicity (ADCC).

If the antibody binds to a site critical for the biological function of a molecule, the activity of the molecule can be neutralized. In this way, specific antibodies can block the binding of a virus or a protozoan to the surface of a cell. Similarly, bacterial and other types of toxins can be bound and neutralized by appropriate antibodies. Moreover, regardless of whether a bound antibody neutralizes its target, the resulting antigen-antibody complex can interact with other defense mechanisms, resulting in destruction and/or clearance of the antigen.

Vaccines are designed to stimulate the immune system to protect against microorganisms such as viruses. When a foreign substance invades the body, the immune system activates certain cells to destroy the invader. This activation of the immune system involves two main types of cells: B cells and T cells. In humoral defense, B cells make antibodies, molecules that attach to and neutralize viruses floating free in the bloodstream, thereby preventing the viruses from infecting other cells. In cell mediated defense, T cells can be helper cells or killer cells. Helper T cells organize the immune response. Killer T cells (known as CD8+CTLs) attack cells infected by viruses.

The purpose of vaccines, then, is to induce immunological memory to accelerate both humoral and cell-mediated responses upon the exposure of a pathogen. The most effective vaccines are those that raise B-cell antibodies against neutralizing epitopes and also stimulate T-cell responses across a broad spectrum of variant strains. It is thought that specific IgA and IgG antibodies, directed mainly at extraviral epitopes are produced by B memory cells after a re-challenge. The re-introduced virus is then eliminated by the forming virus-Ig complexes. CD8+ memory T cells are also induced and kill host cells that are infected with the virus accelerating their elimination. It is thought that these memory T cells (CD4+ and CD8+) are directed against internal viral proteins.

Currently available viral vaccines fall either into 2 categories, those from whole virus or subunit vaccines made from purified viral antigens. The vast majority of viral vaccines at present are whole virus either live or inactivated. Inactivated whole viruses are the easiest preparations as the original virus is grown by normal virus culture methods e.g. tissue culture (Polio for the Salk vaccine), eggs (Influenza), mouse brain (Rabies Semple vaccine). The viruses are then harvested from their respective culture, purified and then chemically inactivated by formalin or B-propiolactone destroying the viral replicative function and further infectivity. Live whole virus are prepared from mutated strains that are attenuated to be almost or completely devoid of pathogenicity but still are capable of inducing a protective immune response.

Introduction of these attenuated strains may multiply in the human host and provide continuous antigenic stimulation over a period of time. There are several approaches to obtain attenuated viral strains for vaccination. One can use a closely related virus that typically uses another animal as the host. The most famous example is Edward Jenner's use of cowpox virus to prevent smallpox. Another methodology is to administer the virus through an unnatural route whereby the virulence of the virus is reduced. This principle is used in the immunization of military recruits against adult respiratory distress syndrome using enterically coated live adenovirus types 4, 7 and 21. Attenuation can also be achieved through genetic screens by passage of the virus in an “unnatural host” or host cell to generate temperature sensitive mutants. The major vaccines used in man and animals have all been derived this way. For example, the 17D vaccine of yellow fever was developed by passage in mice and then chick embryos, polioviruses were passaged in monkey kidney cells.

There are however, significant disadvantages in regards to whole viral vaccines. It is possible that back-mutation(s) of live attenuated virus will result in virulence reversion as in the case of polio Sabin vaccine. Additionally, live vaccines are not appropriate for immunocompromized and pregnant patients. The introduction of some whole virus species are very undesirable as in the case of oncogenic Epstein Barr viruses (EBV) or are capable of extremely dangerous infections like HIV. Further technical problems exist, as some viruses cannot be readily cultivated for example, Hepatitis B and parvovirus.

The development of subunit vaccines from synthetic peptides is steadily proving to be very useful. The best-known example is foot and mouth disease, where protection is achieved by immunizing animals with a linear sequence of 20 amino acids. Synthetic peptide vaccines have many advantages. The peptide antigens are precisely defined and free from unnecessary components, which may be associated with side effects. They can be formulated to be stable for long-term storage and are relatively cheap to manufacture compared to whole virus needing tissue or egg cultivation. In being well defined sequence(s), new amino acid changes due to natural evolution of the virus can be readily accommodated. This ready adaptation would be a great advantage for unstable viruses such as Influenza. Another advantage is that peptide fragments can be derived from oncogenic viruses or those that cannot be readily cultivated.

To combat influenza disease, prophylactic vaccination is the current method using vaccines that induce adaptive humoral immunity against the influenza strains. The current influenza vaccine for that year is then derived from selected viral strains grown in egg based culture systems. However, many viruses are capable of great antigenic variation, and large numbers of serologically distinct strains of these viruses have been identified. As a result, a particular strain of a virus becomes insusceptible to immunity generated in the population by previous infection or vaccination. During the past century, influenza viruses with hemagglutinin (HA) glycoproteins from 3 of the 15 influenza A virus subtypes (H1-H15) emerged from avian or animal hosts to cause worldwide epidemics: in 1918, H1; in 1957, H2 and in 1968, H3 (WHO Memorandum (1980) Bull. W. H. O. 58, 585-591). Attempts to control influenza by vaccination has so far been of limited success and are hindered by continual changes in the major surface antigen of influenza viruses, the hemagglutinin (HA) and neuraminidase (NA), against which neutralizing antibodies are primarily directed (Caton et al. (1982) Cell 31:417; Cox et al. (1983) Bulletin W. H. O. 61:143; Eckert, E. A. (1972) J. Virology 11:183). The influenza viruses have the ability to undergo a high degree of antigenic variation within a short period of time. It is this property of the virus that has made it difficult to control the seasonal outbreaks of influenza throughout the human and animal populations.

Through seralogic and sequencing studies, two types of antigenic variations have been demonstrated in influenza A viruses. Antigenic shift occurs primarily when either HA or NA, or both, are replaced in a new viral strain with a new antigenically novel HA or NA. The occurrence of new subtypes created by antigenic shift usually results in pandemics of infection. Antigenic drift occurs in influenza viruses of a given subtype. Amino acid and nucleotide sequence analysis suggests that antigenic drift occurs through a series of sequential mutations, resulting in amino acid changes in the polypeptide and differences in the antigenicity of the virus. The accumulation of several mutations via antigenic drift eventually results in a subtype able to evade the immune response of a wide number of subjects previously exposed to a similar subtype. In fact, similar new variants have been selected experimentally by passage of viruses in the presence of small amounts of antibodies in mice or chick embryos.

An influenza subunit vaccine that can stimulate CTLs to recognize conserved immunodominant viral epitopes would be of great benefit as it can offer broad heterotypic cross protection to all strains carrying those particular protein sequences and even slight variations thereof. These epitope based vaccines would be expected to elicit protective immunity as activated CTLs would recognize and lyse virus infected cells presenting those epitopes on their cell surface MHC. An additional feature that can be integrated into epitope based vaccines are peptides that stimulate both antibody producing B cells and/or T-helper cells. Furthermore, vaccines using polypeptide sequence permutations that can incorporate the genetic variation of existing strains and evolutionary drift possibilities would also be of great prophylactic benefit as it could stimulate broad pan reactive antibodies against highly variable existing and changing surface proteins.

One of the objectives of subunit vaccines is to provide to antigen presenting cells (APCs), peptide sequences that are efficiently processed and presented by the MHC molecules. The identification of numerous MHC epitopes derived from viral antigens has opened the door to the development of peptide based subunit vaccines. However, not all peptides are sufficiently immuogenic as MHC restriction requires that the epitope amino acid sequence be specifically tailored for that particular MHC class and allele in order for robust epitope binding to occur. Furthermore, another problem with many epitopes derived from native viral proteins, is that they have relatively low inherent MHC affinities. Lower epitope-MHC affinity interactions lead to less complex stability and shortened APC surface presentation. As such, the available CTLs that could potentially recognize the infected cells and respond are not realized. Thus, low MHC affinity epitopes are not always the ideal vaccine candidates.

### SUMMARY OF THE INVENTION

The invention includes, in one aspect, a method of producing viral antigens for use in a vaccine or therapeutic composition against infection by a virus having a plurality of subtypes whose protein sequences for at least one viral protein or polyprotein are known. The method is applicable to viruses having a plurality of subtypes whose protein sequences for at least one viral protein or polyprotein are known. Epitope regions of viral protein are selected that (i) are known or predicted to bind to MHC proteins in host antigen-presenting cells, and thereafter (ii) the epitope's amino acid sequence are systematically varied, e.g., by Look-Through Mutagenesis and/or combinatorial mutagenesis, (iii) and then tested for enhanced binding to MHC proteins.

In one embodiment, the MHC-binding protein-sequence regions selected for mutagenesis are M2 ion channel peptides selected from the group consisting of SEQ ID NOS: 16 and 17.

In another embodiment, the MHC-binding protein-sequence regions selected for mutagenesis are NS1 peptides selected from the group consisting of SEQ ID NOS: 34 and 35.

The Look Through Mutagenesis in the method may be carried out at each epitope amino-acid position, whereby replacement substitutions by a set of amino acids that collectively, have properties representative of an entire set of natural amino acids. Look Through Mutagenesis may either be performed at MHC epitope regions where none to little variations occur between the known viral subtype sequences.

In another embodiment, the mutagenesis step in the method is carried out by making combinatorial permutations of the amino acid variations that occur within the plurality of subtypes for that region.

Also disclosed is a vaccine or therapeutic composition for use against infection by an influenza virus. In one embodiment, the composition includes one or more peptides selected from the group consisting of M2 peptide fragments identified by SEQ ID NOS: 16-17, 54-63 or 89-90. In another embodiment of the invention, the composition includes peptides selected from the group consisting of NS1 peptide fragments identified by SEQ ID NOS: 34-35 or from the group consisting of NS1 peptide fragments identified by SEQ ID NOS: 78-85. In yet another embodiment of the invention, the composition includes peptides selected from the group consisting of M1 peptide fragments identified by SEQ ID NOS: 86-88.

These and other objects and features of the invention will become more fully apparent when the following detailed description of the invention is read in conjunction with the accompany drawings.

### BRIEF DESCRIPTION OF THE DRAWINGS

FIGS. 1A-1C depict influenza viruses A, B, and C, respectively, showing the eight peptides encoded by each virus;

FIG. 2 is a flowchart of steps in the method of the invention, including (i) aligning a set of peptide sequences in a plurality of subtypes for a selected virus, (ii) selecting regions of MHC binding, (iii) identifying regions of moderate amino-acid variation, (iv) selecting sets of peptide regions for look-through mutagenesis, (v) steps in generating modified peptides by look-through mutagenesis, and (vi) selecting those modified peptides having the highest binding affinity for an MHC protein;

FIGS. 3A-3C show alignment of the M2 protein from 15 subtypes of avian flu virus, an MHCI binding region having the sequence DPLVIAASI with aligned positions of M2 variability for constructing a combinatorial variant vaccine (3B), and an MHCII binding region having the sequence RNGWECRCSDSSDPL with aligned positions of M2 variability for constructing a combinatorial variant vaccine (3C);

FIGS. 4A-4C show alignment of the NS1 protein from 15 subtypes of avian flu virus, an MHCI binding region having the sequence IFDRLETLI with aligned positions of NS1 variability for constructing a combinatorial variant vaccine (4B), and an MHCII binding region having the sequence DEALKMTIASVPASR with aligned positions of NSI variability for constructing a combinatorial variant vaccine (4C);

FIGS. 5A-5I show sequence variations for nine substituted amino acids in LTM peptide variations for the M2 peptide region DPLVIAASI; and

FIGS. 6A-6I show corresponding DNA coding sequences for the LTM peptide variants shown in FIGS. 5A-5I, respectively.

FIG. 7: Enumeration of the relative amino acid frequencies at each position in the NS1₁₂₂₋₁₃₀ epitope region after comparative alignments of ˜1300 Influenza strains. NS1₁₂₂₋₁₃₀ positions 123, 124, 125, 127 and 129 illustrate various amino acids occurring at particular frequencies (identified by arrows). variability with M and V, position 127 with T and R, and position 129 with I and T amino acids respectively. Two of these positions, were represented at greater than the 15% threshold frequency value and included for further combinatorial mutagenesis.

FIG. 8: Enumeration of the relative amino acid frequency at each position in the M1₅₈₋₆₆ peptide GILGFVFTL epitope region after comparative alignments of ˜1300 Influenza strains. Influenza M1 epitope demonstrated that the consensus GILGFVFTL amino acid sequence within each position was greater than 99% threshold frequency. The conserved GILGFVFTL sequence and is considered “fixed” and candidate for Look Through Mutagenesis not a candidate for combinatorial mutagenesis.

FIG. 9: Enumeration of the relative amino acid frequency at each position in the NS1₂₃₂₋₂₄₀ epitope region after comparative alignments of ˜1300 Influenza strains.

FIG. 10: Enumeration of the relative amino acid frequency at each position in the HA₃₀₆₋₃₁₈ epitope region after comparative alignments of ˜1300 Influenza strains.

FIG. 11: shows the coding DNA sequence for the Combinatorial Multi-Epitope vaccine constructs.

FIG. 12: shows the Combinatorial Multi-Epitope vaccine constructs constructed as a contiguous linear peptide. The individual NS1, M1 and M2 epitopes are flanked by intervening lysine and/or arginine (K/R) proteolytic cleavage sites. The upper sequence (CME1) illustrates one version of the construct with the eight variant NS1 epitopes linked together then followed by the three M1 optimized for HLA-0201 and two M2 variant epitopes optimized for HLA-B44. The lower (CME2) illustrates the a version where the first four variant NS1 epitopes are separated by the three M1 and two M2 epitopes and then followed by the remaining four variant NS1 epitopes.

### DETAILED DESCRIPTION OF THE INVENTION

### I. Viral Targets

The method of the invention can be applied to any pathogenic viral target for which the sequences for a plurality of virus subtypes are known. For purposes of illustration, the method will be applied below to producing novel vaccine or therapeutic peptide compositions against influenza viruses, and in particular, the avian influenza virus, an influenza virus. It will be appreciated from the description how the method can be applied to other pathogenic viruses of interest.

A. Influenza Virus Structure and Organization

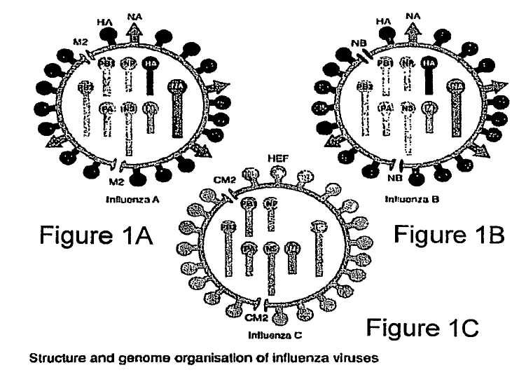

The influenza virion is generally rounded but may be long and filamentous. A single-stranded RNA genome is closely associated with a helical nucleoprotein (NP), and is present in eight separate segments of ribonucleoprotein (RNP), each of which has to be present for successful replication. The segmented genome is enclosed within an outer lipoprotein envelope. An antigenic protein called the matrix protein (MP 1) lines the inside of the envelope and is chemically bound to the RNP. The envelope carries two types of protruding spikes. One is a box-shaped protein, called the neuraminidase (NA), of which there are nine major antigenic types, and which has enzymatic properties as the name implies. The other type of envelope spike is a trimeric protein called the haemagglutinin (HA) (illustrated on the right) of which there are 13 major antigenic types. The haemagglutinin functions during attachment of the virus particle to the cell membrane, and can combine with specific receptors on a variety of cells including red blood cells. The composition and distribution of viral proteins in Influenza A, B, and C viruses are seen in FIGS. 1A-1C, respectively.

With reference to the figures, influenza viruses are composed of eight segments of single stranded RNA of negative polarity, totaling approximately 14 kilobases, encode for at least 10 viral proteins. The three viral polymerases (PB1, PB2 and PA) are encoded by approximately half of the total genome by RNA segments 1, 2 and 3 respectively. RNA segment 5 encodes the NP protein. The three-polymerase subunits, the NP and the vRNA then associate as virions in infected cells in the form of viral ribonucleoprotein particles (vRNPs). RNA segments 4 and 6 encode for the HA and NA genes, respectively. The two smallest RNA segments (7 and 8) encode two genes each with overlapping reading frames, which are generated by splicing of the co-linear mRNA molecules. In addition to M1, RNA segment 7 encodes for the M2 protein, which has ion channel activity and is embedded in the viral envelope. Segment 8 encodes for NS1, a nonstructural protein that blocks the host's antiviral response, and NS2 or NEP that participates in the assembly of virus particles.

The Influenza viruses are enclosed in a lipid envelope that is acquired in the final step of virus assembly. The viruses bud from the host cell membranes where the virally encoded glycoproteins: HA and NA have accumulated. After budding, the Influenza envelope is spiked with HA and is the most abundant protein on the virus surface. In subsequent infection of new host cells, HA plays an important role in virus recognition, attachment and membrane fusion. After host cell receptor attachment, the virus is then internalized by endocytosis. Acidification of the endosome then leads to conformational changes of HA protein fusing the viral and the endosomal membranes. Endosomal acidification also activates the ion channel activity of influenza matrix protein 2 (M2) whereupon an inward current of protons into the virion's interior. Influenza virus protein M2 is a small (96-amino acid) integral membrane protein that spans the cell membrane once and exists as a disulfide-linked homotetramer. The M2 protein acts as an ion channel during the endosome uncoating process permitting a flow of protons into the interior of virus particles which then disrupts protein-protein interactions. It is these disruptions that triggers the disassembly of matrix protein 1 (M1) from the vRNPs.

The released vRNPs, composed of viral RNA (vRNA) and nucleocapsid proteins (NP), are then transported to the nucleus for virus transcription and replication. Two different populations of positive sense RNAs are synthesized from vRNA templates: messenger RNAs (mRNAs) and complementary RNAs (cRNAs). The first step is the synthesis and transcription of cRNA representing full-length copies of vRNAs. The virus carries its' own RNA replicase complex (PB1, PB2 and PBa) as the host cell lacks protein(s) capable of performing this function. Viral mRNAs are then primed by 5′ capping fragments and polyadenylated for export and proper protein translation in the cytoplasm.

The second step in viral replication is the synthesis of progeny vRNA genomes from cRNAs templates. As the infection cycle progresses, when sufficient M1 matrix protein and nucleocapsid (NP) have been produced, the newly synthesized vRNPs are then exported out of the nucleus and assembled into full virus particles. The final assembly steps occur at the plasma membrane incorporating the newly synthesized HA, NA, and M2 proteins. HA and NA are present as homotrimers and homotetramers, respectively on the viral envelope. Within the envelope, M1 and NP proteins protect the vRNA.

The mature HA homotrimer is initially processed from a single polypeptide precursor (HA0). HA0 is subsequently cleaved into the subunits: HA1 and HA2. Both HA1 and HA2 subunits are glycosylated and are linked by a single disulphide bond between them. At the N-terminal end of the HA2 chain is the fusion peptide which is critical for subsequent membrane fusion events that lead to infection. The HA0 requires post translational cleavage by host proteases before it is functional and virus particles are infectious. Most HA proteins have some sequence consensus motif (R-X-R/K-R*-G-L-F) found within the HA1-HA2 connecting peptide. Viral pathogenicity is correlated with cleavage susceptibility. For example, The HA0 precursor proteins of low virulence avian influenza viruses have a single arginine at the cleavage site and are limited to cleavage by host proteases such as trypsin-like enzymes and are thus restricted to replication at sites in the host where such enzymes are found, i.e. the respiratory and intestinal tracts. In contrast, highly pathogenic avian influenza viruses possess multiple basic amino acids [arginine and lysine] at their HA0 cleavage sites and appear to be cleavable by a ubiquitous protease[s] such as subtilisin-related endoproteases.

B. Vaccine Proteins

Three influenza-A proteins have been selected as vaccine candidates in the present method: the M2 ion channel protein, the NS1 non-structural protein, and the HA0 precursor of HA. However, it will be appreciated that other virion proteins, including NA and HA, may be employed in the method for producing enhanced vaccine or therapeutic peptides for vaccination or therapy against influenza viruses.

M2 ion-channel protein. The life cycles of both major strains of influenza virus, A and B, are quite similar. The influenza virus is bound by a membrane taken from the plasma membrane of an infected cell. The M2 protein is present in this membrane, and it's main role is to provide for acidification of the interior of the virion while it is contained within the acidic endosome. Only by acidification prior to fusion of the virion membrane with the endosomal membrane is it possible for the viral RNA to be released from the viral matrix protein (the process of uncoating). Uncoating is made possible by relaxation of interactions between the ribonuclear proteins (e.g., RNA-dependent RNA polymerase not encoded by the host cell genome) and the matrix protein. A second role for the M2 protein of some subtypes of virus is to shunt the pH gradient of the Golgi apparatus, preventing premature conformational change of the hemagglutinin (HA). M2 is relatively small, about 0 KDaltons, has a portion that is exposed on the virion surface, and unlike its much larger cirus-surface neighbors, NA and HA, is relatively invariant in its amino acid sequences across the subtypes for any influenza virus.

NS1 protein. A decisive factor for the efficient replication of influenza and several other viruses is the ability to inhibit in their hosts the expression of the antiviral cytokines alpha interferon (IFN-) and IFN-β. Recent analyses have established that the nonstructural NS1 protein of influenza A virus (termed NS1-A) is a major force that antagonizes activation of PKR and the expression of early defense genes, including IFN-β.

NS1-A is a multifunctional 26-kDa protein that has been reported to bind to single- and double-stranded RNAs, to inhibit the polyadenylation and splicing of cellular pre-mRNAs, and to enhance translation. Notably, either the ability to sequester virus-derived dsRNAs and thereby to reduce the signaling for IFN gene activation or the blockade of early antiviral defense transcripts on a posttranscriptional level have been suggested to explain the IFN-antagonistic function of the NS1-A protein.

M1 protein. M1 protein of influenza A virus has multiple regulatory functions during the infectious cycle, which include mediation of nuclear export of viral ribonucleoproteins, inhibition of viral transcription and a crucial role in virus assembly and budding.

NA protein. Neuraminidase spans the lipid bilayer and forms a tetrameric structure as a viral surface glycoprotein. NA acts as an enzyme, cleaving sialic acid from the HA molecule, from other NA molecules and from glycoproteins and glycolipids at the cell surface.

HA protein. The mature HA homotrimer is initially processed from a single polypeptide precursor (HA0). HA0 is subsequently cleaved into the subunits: HA1 and HA2. Spanning the lipid membrane, both HA1 and HA2 subunits are glycosylated and are linked by a single disulphide bond between them. At the N-terminal end of the HA2 chain is the fusion peptide which is critical for subsequent membrane fusion events that lead to infection. HA serves as a receptor by binding to sialic acid (N-acetyl-neuraminic acid) and induces penetration of the interior of the virus particle.

The HA0 requires post translational cleavage by host proteases before it is functional and virus particles are infectious. Most HA proteins have some sequence consensus motif (R-X-R/K-R*-G-L-F) found within the HA1HA2 connecting peptide. Viral pathogenicity is correlated with cleavage susceptibility. For example, The HA0 precursor proteins of low virulence avian influenza viruses have a single arginine at the cleavage site and are limited to cleavage by host proteases such as trypsin-like enzymes and are thus restricted to replication at sites in the host where such enzymes are found, i.e. the respiratory and intestinal tracts. In contrast, highly pathogenic avian influenza viruses possess multiple basic amino acids [arginine and lysine] at their HA0 cleavage sites and appear to be cleavable by a ubiquitous protease[s] such as subtilisin-related endoproteases.

### II. Overview of Method

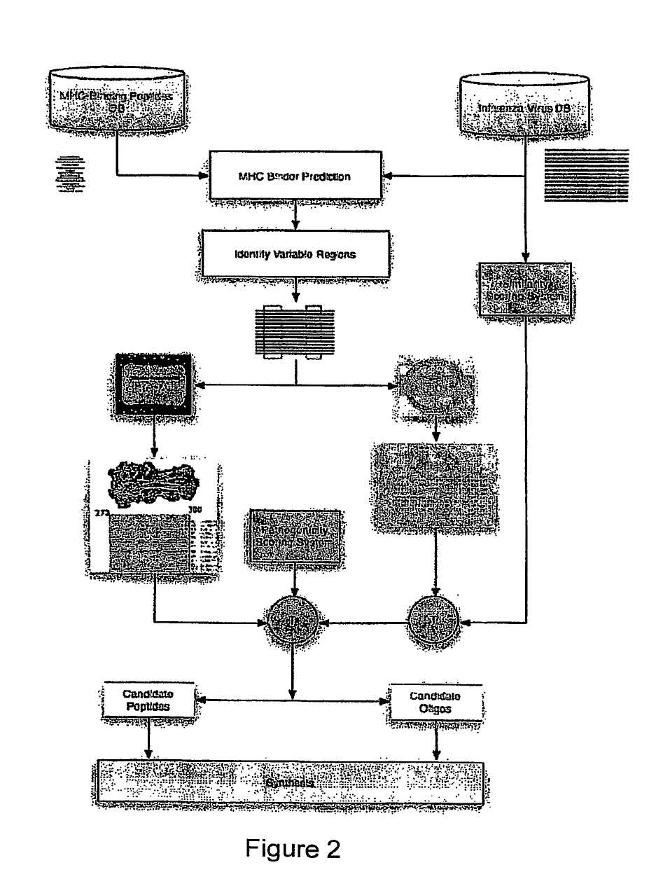

FIG. 2 is an overview of the steps of the method for producing vaccine or therapeutic peptides in accordance with the invention. As seen at the top of the figure, the method employs information from two databases 10, 12. Database 10 contains the amino acid sequences of known and predicted human MHC I and MHC II proteins, such as indicated at 11 in the figure. These sequence information is available, for example from the SYPEITHI database, which can be accessed at (www.SYPEITHI.de). Other MHC databases include the Jenpep (www.jenner.ac.uk/JenPep/), IMGT (http://imgt.cines.fr/), NHLAPred (http://www.imtech.res.in/raghava/nhlapred/neural.html), ProPredl (http://www.imtech.res.in/raghava/propred/), and EPIPREDICT (http://www.epipredict.de/).

Database 12 contains the genomic sequences of each of the subtypes of a given virus that has been sequenced to date, and the corresponding amino acid sequences of at least some of the viral proteins, as indicated at 15 in the figure. This sequence information is available, for example from the Influenza Sequence Database database at Los Alamos National Laboratory, which can be accessed at (www.flu.lanl.gov). Other Influenza sequence databases for analysis include The Institute for Genomic Research (www.tigr.org), UNIPROT (www.ebi.uniprot.org/database), and National Center for Biotechnology Information (www.NCBI.nlm.nih.gov).

The MHC-binding protein sequences and virus-subtype sequences are employed in identifying two special regions of one or more selected proteins from a given pathogenic virus of interest. As noted above, and for purposes of illustration only, the method will be illustrated herein for identifying these protein regions for the M2 ion channel protein and NS1 non-structural protein of avian flu virus.

The first regions of interest for a given viral protein are those regions that have or are predicted to have high binding affinity for a human MHC I or MHC II binding protein. Where such a region has already been experimentally verified, it may be so identified in the virus-sequence database or disclosed in a scientific publication. More typically, the virus-protein region of interest will be found using a standard program for predicting binding between a given MHC I or II protein sequence, and a segment of the viral protein of interest. A number of binding-prediction programs capable of this determination are available, such as the SYPEITHI program. The program may be carried out on a single subtype sequence for a selected protein, or may be carried out on each of the subtype sequences, to ensure that the regions identified are common to all or a majority of the subtypes.

The clinical effectiveness of influenza vaccination is dependent on several factors, including the vaccine immunogen, dosage and the closeness of the match between the vaccine and circulating influenza virus strains. However, a significant component in the immunogical response is partially modulated by the individuals Major Histocompatibility Complex (MHC) alleles (also known as Human Leukocyte Antigens HLA in humans). Normally functioning cells process and continually present on their surface, short peptides bound to Class I and II MHC molecules. When the presented peptides are the products of an invading foreign genome, such as a virus that has infected the cell, the loaded extracelluluar MHC complex is then recognized by cytotoxic T cells, to trigger a series of events which terminates in apoptotic host cell death. The ability to determine which protein segments will bind particular MHC molecules with high affinity is therefore of importance for the development of peptide vaccines.

The major histocompatability complex (MHC) is a large genomic region that contains a series of linked genes that have critical roles the presentation of antigens to T-cells and in the recognition and discrimination between “self” and “non-self” molecules by T-cells. In humans, the MHC spans approximately 4 Mb and is located on the short arm of chromosome 6. The most extensively studied genes in the human MHC are the genes encoding the human leukocyte antigen (HLA) proteins. These proteins anchor in cell membranes and present antigenic peptides to the T lymphocytes resulting in the initiation of specific immune responses. The HLA proteins are encoded by two classes of genes, HLA class I and HLA class II. Class I HLA genes include HLA-A, HLA-B and HLA-C, and class II HLA genes include HLA-DR, HLA-DQ, HLA-DQB1, and HLA-DP.

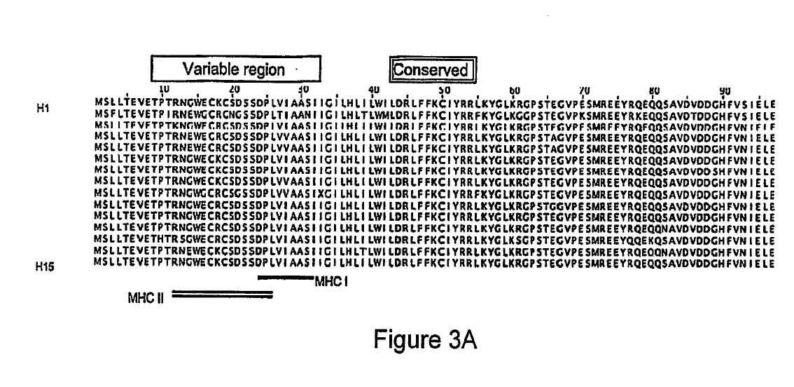

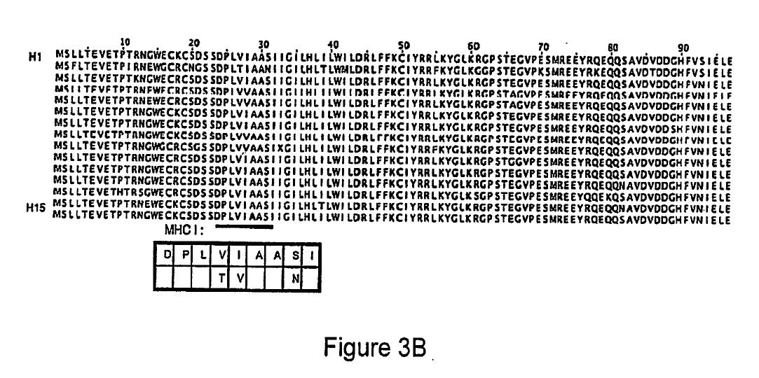

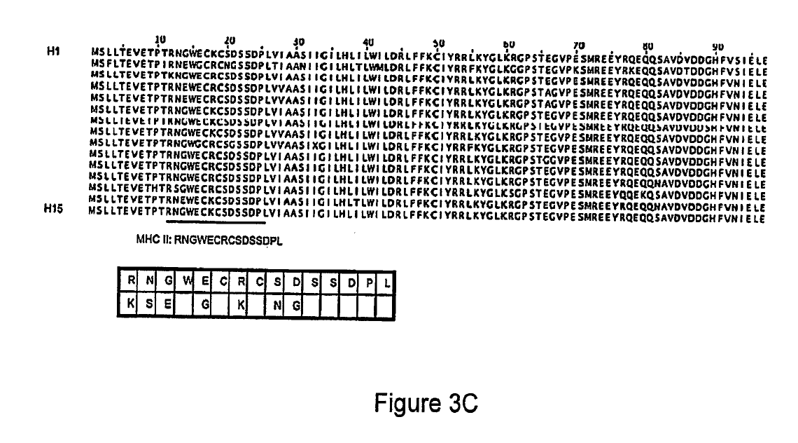

Once the regions of high MHC binding are identified, the method now looks for the high MHC binding regions that also show a moderate degree of amino acid variability among the subtype sequences for that viral protein. Typically, this means that the region will contain a plurality (2 or more) of residue positions at which a small number of amino acid variations, e.g., 2-6 different amino acids occur, and a plurality of position that are invariant or substantially invariant in amino acid substitution. In the analysis described below for the M2 protein, with respect to FIG. 3A, the high MHC I binding region 25-33 identified by the sequence DPLVIAASI contains three out of nine positions with minor amino acid substitutions (2 amino acids at each position). Similarly, the MHC II binding regions 12-26 identified by RNGWECRCSDSSDPL contains 7 position with amino acid variability and eight with no variability. Typically, the region of interest will be between 8-16 amino acids in length, and have amino acid variations in about half or less of the segment. FIG. 2 shows at 18 the aligned sequences for a given protein and two regions 18a, 18b identified by the two criteria above.

The protein regions identified above on the basis of MHC binding and moderate amino acid variability are used, in accordance with various aspects of the invention, to form combinatorial peptides with enhanced binding, as indicated at 20 in the figures, or to form single-substitution peptides with enhanced binding activity, as indicated at 22 in the figures. In the combinatorial methods, peptides containing various permutations of the amino acid variations observed in nature are produced. For example, in the above M2-protein MHCI binding sequence, the combinatorial peptides would include the 23 different sequence containing all combinations of the amino acid sequence variations at the 4th, 5th, and 8th position in the peptide region. A set of combinatorial peptides is show schematically at 24 in the figure.

The purpose of look-through mutagenesis (LTM) is to introduce a selected substitution at each of target mutation positions in a region of a polypeptide, e.g., the high-binding affinity region identified above. Unlike combinatorial methods, LTM confines substitutions to a single selected position. Typically in the method, a subset of natural amino acids, e.g., nine different amino acids that represent the physico-chemical spectrum of properties of natural amino acids, are selected. For each of these amino acids, peptides containing a single substitution of that amino acid at each position are prepared, e.g., using modified coding sequences in an in vitro cell-free synthesis system. These steps are illustrated below for the MHC I and II binding regions of the avian-flu M2 and NS1 proteins. A set of LTM peptide variants is shown schematically at 30 in the figure.

The combinatorial and LTM peptides are prepared and tested for enhanced MHC I and II binding as described below, as indicated at 28 and 32 in FIG. 2. From this testing, candidate peptides are identified at 34, and corresponding oligo coding sequences at 36. The peptides are synthesized and tested for vaccine and/or therapeutic efficacy. As indicated at 38.

The clinical effectiveness of influenza vaccination is dependent on several factors, including the vaccine immunogen, dosage and the closeness of the match between the vaccine and circulating influenza virus strains. However, a significant component in the immunogical response is partially modulated by the individuals Major Histocompatibility Complex (MHC) alleles (also known as Human Leukocyte Antigens HLA in humans). Normally functioning cells process and continually present on their surface, short peptides bound to Class I and II MHC molecules. When the presented peptides are the products of an invading foreign genome, such as a virus that has infected the cell, the loaded extracelluluar MHC complex is then recognized by cytotoxic T cells, to trigger a series of events which terminates in apoptotic host cell death. The ability to determine which protein segments will bind particular MHC molecules with high affinity is therefore of importance for the development of peptide vaccines.

The major histocompatability complex (MHC) is a large genomic region that contains a series of linked genes that have critical roles the presentation of antigens to T-cells and in the recognition and discrimination between “self” and “non-self” molecules by T-cells. In humans, the MHC spans approximately 4 Mb and is located on the short arm of chromosome 6. The most extensively studied genes in the human MHC are the genes encoding the human leukocyte antigen (HLA) proteins. These proteins anchor in cell membranes and present antigenic peptides to the T lymphocytes resulting in the initiation of specific immune responses. The HLA proteins are encoded by two classes of genes, HLA class I and HLA class II. Class I HLA genes include HLA-A, HLA-B and HLA-C, and class II HLA genes include HLA-DR, HLA-DQ, HLA-DQB1, and HLA-DP.

The present invention includes, in one aspect, the building and annotation of a relational immunogenetic database to identify regions of moderately high sequence conservation from the Influenza intraviral M2 and NS1 proteins. The immunogenetic database will first identify conserved amino acid regions within M2 and NS1 between different HA1-HA-15 subtypes of Influenza. These conserved regions are then interrelated with both MHC I and II peptide prediction presentation scores. Within each identified region the amino acid variation allows the generation of combinatorial sequences (see other patent). A consensus sequence can also be derived from each candidate region. These identified sequences are then further optimized, in silico, using Look Through Mutagenesis and combinatorial mutagenesis for enhanced MHC binding.

SEQ ID NO: 17 M2: (residues 12 to 26);
RNGWECRCSDSSDPL

From the above sequence we have calculated the following amino acid occurrence:

Aa₁ Aa₂ Aa₃ W Aa₄C Aa₅ C Aa₆Aa₇ SSDPL

Where Xaa1 is selected from the group consisting of R and K;

Xaa₂ is selected from the group consisting of N and S;

Xaa₃ is selected from the group consisting of G and E;

Xaa₄ is selected from the group consisting of E, and G;

Xaa₅ is selected from the group consisting of R and K;

Xaa₆ is selected from the group consisting of S and N;

Xaa₇ is selected from the group consisting of D and G;

SEQ ID NO: 16 M2: (residues 24 to 32);
DPLVIAASI

From the above sequence we have calculated the following amino acid occurrence:

D P L Aa₁ Aa₂ A A Aa₃ I

Where Xaa₁ is selected from the group consisting of V and T;

Xaa₂ is selected from the group consisting of V and I;

Xaa₃ is selected from the group consisting of S and N;

SEQ ID NO: 35 NS1: (residues 74 to 88);
DEALKMTIASVPASR

From the above sequence we have calculated the following amino acid occurrence:

D Aa₁ Aa₂ Aa₃ K Aa₄ Aa₅ Aa₆Aa₇ S Aa₈ Aa₉ A Aa₁₀ R

Where Xaa₁ is selected from the group consisting of E and K;

Xaa₂ is selected from the group consisting of A and I;

Xaa₃ is selected from the group consisting of L and F;

Xaa₄ is selected from the group consisting of M, and I;

Xaa₅ is selected from the group consisting of T, N and A;

Xaa₆ is selected from the group consisting of I and M;

Xaa₇ is selected from the group consisting of A and T;

Xaa₈ is selected from the group consisting of V, I and L

Xaa₉ is selected from the group consisting of P and L

Xaa₁₀ is selected from the group consisting of S and R

SEQ ID NO: 34

NS1: (residues 137 to 145); IFDRLETLI

From the above sequence we have calculated the following amino acid occurrence:

I F Aa₁ R L E Aa₂ L Aa₃

Where Xaa₁ is selected from the group consisting of D, G and N;

Xaa₂ is selected from the group consisting of T, A, and N;

Xaa₃ is selected from the group consisting of I and T;

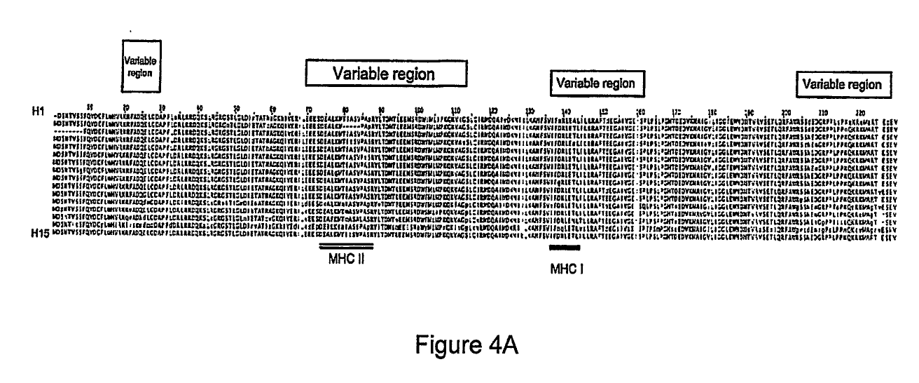

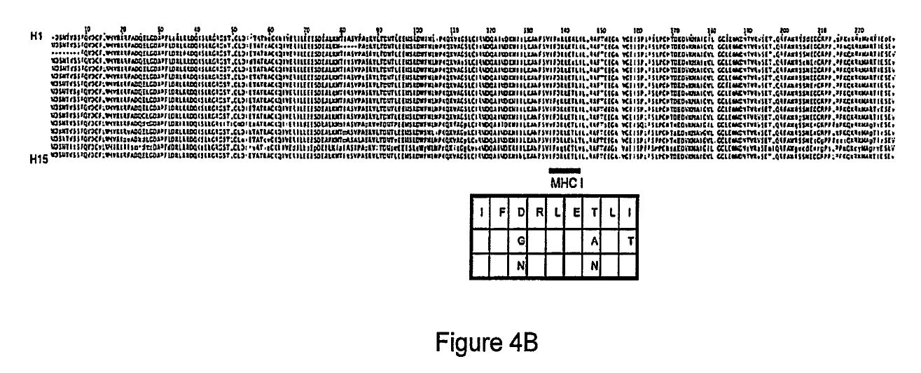

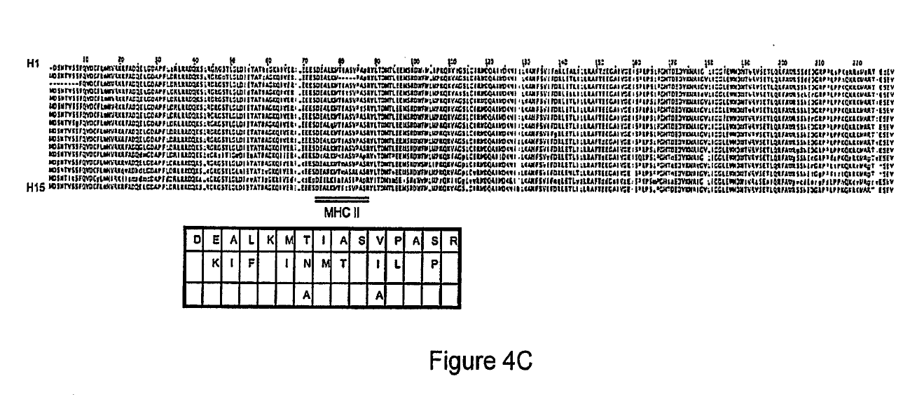

H3 in FIG. 4a SEQ ID NO:20 M2: Full length query Influenza M2 protein;

MSLLTEVETPTRNGWECKGSDSSDPLVIAASIIGILHLILWILDRLFFKC
IYRRLKYGLKRGPSTEGVPESMREEYRQEQQSAVDVDDGHFVSIELE

SEQ ID NO: 31 NS1: Full length query Influenza NS1 protein;

MDPNTVSSFQVDCFLWHVRKRVADQELGDAPFLDRLRRDQKSLRGRGSTL
GLDIKTATRAGKQIVERILKEESDEALKMTMASVPASRYLTDMTLEEMSR
DWSMLKQKVAGPLCIRMDQAIMDKNIILKANFSVIFDRLETLILLRAFTE
EGAIVGEISPLPSLPGHTAEDVKNAVGVLIGGLEWNDNTVRVSETLQRFA
WRSSNENGRPPLTPKQKREMAGTIRSEV

SEQ ID NOS: 53-57 Look Trough Mutagenesis M2 ion channel peptides selected that are predicted to be improved for MHC I binding.

DPLVIAARI
DPLVIAASR
DPLVSAASI
DPLVIAAYI
DPLVIAASY

SEQ ID NOS: 58-63: Combinatorial Peptides from influenza M2 that are predicted to be improved for MHC I binding;

DPLTIAASI
DPLVVAASI
DPLTVAASI
DPLTIAANI
DPLVVAANI
DPLTVAANI

Due to antigenic shift and drift, it has become therefore necessary to identify regions that conserved between influenza A and B strains to derive a broadly cross protective vaccine akin to some natural infections. This will allow protection from infection challenges with either drift viruses within a subtype or shift subtypes (Tamura S, Tanimoto T, Kurata T. Mechanisms of broad cross-protection provided by influenza virus infection and their application to vaccines. Jpn J Infect Dis. 2005 August; 58(4):195-207). For example, some recent studies found that human influenza vaccines based on the extracellular domain of influenza M2 protein (M2e) induced broad-spectrum protective immunity in various antigen constructs (REFS). Particular M2 peptide can be selected for immunization as it is highly conserved among many different influenza A strains, including avian flu strains, compared to other influenza A membrane proteins.

### III. Identification of Candidate Vaccine Regions

Although a large amount of information on the viral sequence, function, and pathogenicity is becoming available, it is often difficult to understand the relationship between them. We have created an integrated relational database that search allows integration and cross referencing of the 1—human genetic MHC allele peptide binding preferences, 2—Influenza sequence, and 3—Influenza epidemiological data databases. Current Influenza sequence databases include the Los Alamos www.flu.lanl.gov/ (LANL: Macken, C., Lu, H., Goodman, J., & Boykin, L., “The value of a database in surveillance and vaccine selection.” in Options for the Control of Influenza IV. A. D. M. E. Osterhaus, N. Cox & A. W. Hampson (Eds.) Amsterdam: Elsevier Science, 2001, 103-106) and NIH NCBI databases (others).

The amino acid sequences from the Influenza A, M2 and NS1 protein for 15 known subtypes were aligned by sequence homology, as shown in FIGS. 2, 3 and 4 respectively. There are a variety of alignment algorithms to compare multiple amino acid sequences (MSF, PIR, Clustal W, etc). The basic practice of alignments lining up two or more sequences is simply to achieve maximal levels of identity (and/or similarity of amino acid functional groups) for the purpose of assessing the degree of similarity and the possibility of homology. Shared conserved amino acid regions will typically emerge after multiple alignments and if necessary, the use of introduced gaps to account for length disparities between entries. These shared blocks are usually continuous amino acid stretches indicative of highly conserved protein function. The areas between conserved blocks are usually more variable regions not under selective functional pressure. In these variable stretches, there may be a range in the frequency of occurrence of other substituted amino acids at their respective aligned positions.

One of the selective steps in identifying potential Influenza A HA0, M2 and NS1 peptide immunogens is choosing sequences that are most likely to be presented by a given MHC complex. Empirical studies have shown that only one in about 200 introduced peptides will bind to a given MHC complex. A further complication in picking a set of peptide immunogens is that a very large number of different MHC alleles exist each with a highly selective peptide binding specificity. Therefore, one must pick the MHC allele(s) the vaccine is being designed for. The binding motif for a given MHC class I complex is in most cases 9 amino acids long. These MHC I motifs are characterized by a strong amino acid preference at the P2 and P9 positions and are termed anchor positions. Crystal structural models of peptide bound MHC molecules are available confirming the identification of the deep “anchor” sequences within the groove and flanking sequences MHC processing. For example, in the influenza HA T-cell epitope (307-319), it has been determined that a single tyrosine at 308 is required for binding to HLA-DR1 molecule. A range of synthetic peptides with tyrosines at position 308 (P2) and lysines at 315 (P9) were found to bind DR1 as well as the native peptide.

For computational vaccinology, prediction of MHC-peptides can be divided into two groups: sequence based and structure based methods. Some of the latter algorithms (www.jenner.ac.uk/JenPep) are based on (i) the additive methods and/or (ii) a 3D-Quantitative Structure Activity Relationships (3D-QSAR), and (ii) based on Comparative Molecular Similarity Indices Analysis (CoMSIA). These algorithms will also incorporate quantitative data on peptide binding to transmembrane peptide transporter (TAP) and contain an annotated list of T-cell epitopes. A number of sequence based algorithms, such as the SYFPEITHI MHC database (http://www.syfpeithi.de) contain a tremendous compilation MHC-presented peptides and several thousand naturally processed ligands peptide. These databases describe allelic MHC ligand specificity and their MHC binding motifs.

Allele specific sequence motifs can be identified by studying the frequencies of amino acids in different positions of identified MHC-peptides. For example, the peptides that bind to HLA-A*0201 are often 9 amino acids long (nonamers), and frequently have two anchor residues, a Lysine in position 2 and a Valine in position 9. Besides the anchor residues, there are also weaker preferences for specific amino acids in other positions. These types of HLA-preferred sequence patterns are then implemented in a simple prediction method. One method to include this information is to use a profile, where a score is given for each type of amino acid in each position. The sum of the scores for a given peptide is then used to make predictions such as the SYFPEITHI prediction method.

These databases have promoted the development of bioinformatic tools that predict peptide-MHC binding allowing high throughput in silico design and screening of potential epitope vaccine candidates. Therefore, a viral genome can then computationally searched for peptides with the likelihood of high MHC binding affinity.

A.i. Identification and Generation of M2 Binding to MHC I and II

The SYFPEITHI MHC database (http://www.syfpeithi.de) was queried with the full length M2 protein from Influenza A virus H2N3 (Genbank ID: gi|13182926|gb|AAK14988.1|AF231361_{—}1) using the following sequences:

MSLLTEVETPTRNGWECKCSDSSDPLVIAASIIGILHLILWILDRLFFKC

IYRRLKYGLKRGPSTEGVPESMREEYRQEQQSAVDVDDGHFVSIELE

The program searches along the entire length of the query protein in step-wise fashion shifting the query window one amino acid at a time. Sub-divided query windows are octamers (8-mers) nonamers (9-mers), decamers (10-mers) and 15-mers in length. Each query window then yields a “predictive score” for that particular HLA allele. In our M2 case, the SYFPEITHI prediction values demonstrated that some of the modeled HLA alleles gave a positive score for the likelihood of binding (See Example 1 of the output format). We then examined all MHC II binding peptides with a SYFPEITHI prediction score greater than 20 and determined their location in the M2 full length. Areas of moderate amino acid sequence variability were identified by visual inspection of the aligned sequence. Conserved and variable regions are determined by both the number of substitutions that occur within a given length of the aligned peptide and also the number of occurring substitutions that are present in the examined isolates for that aligned position.

### MHC II Binding Regions:

Beginning at M2 position 37 and ending at position 51, the highest scoring peptide sequence “HLILWILDRLFFKCI” was not found in a region of moderate sequence variability (See FIG. 3a). Within this region, there were only two positions that incurred substitutions at position 39 and 42. Furthermore, at positions 39 and 42, there was only one other alternative amino threonine and methionine respectively. Due to this limited positional and alternative amino acid variability, we therefore considered this to be a “conserved” region. The next highest scoring peptide, beginning at position 40, LWILDRLFFKCIYRR” returning a SYFPEITHI score of 25 was also found to be within this region of high sequence conservation. However, the third peptide RNEWECRCSDSSDPL began at position 12 and returned a SYFPEITHI score of 24 and was localized in the M2 variable region. The H1-H15 isolates alignments of M2 sequences for this region illustrated that there were a moderate amount of amino acid changes between isolates from positions 12 to 26. Seven of the fifteen positions were found to be variable and compared to the consensus sequence, there were nine alternative amino acids exhibited by the strains in the analysis (see FIG. 3c).

A.ii. Generation of Combinatorial Peptides for M2 MHC II to Binding Region

The aligned table below illustrates the consensus sequence (underlined amino acids) for the M2 region from positions 12 to 26. Below each consensus amino acid is the amino acid variability for that M2 position as exhibited by the other examined strains.

| | | | | | | | | | | | | | | | | |
|---|---|---|---|---|---|---|---|---|---|---|---|---|---|---|---|---|
| 12 | R | N | G | W | E | C | R | C | S | D | S | S | D | P | L | 26 |

| | | | | | | |
|---|---|---|---|---|---|---|
| K | S | E | G | K | N | G |

This positional sequence variability then directs the possible combinatorial variants derived from the RNEWECRCSDSSDPL MHC II peptide. There are in total 128 combinatorial possibilities and listed below:

| | | | | | |
|---|---|---|---|---|---|
| L | RNGWECRCSDSSDP | L | RNGWGCRKSDGSDP | L | RNEWGCRCSDSSDP |
| L | RNGWECRCSDGSDP | L | RNGWGCRKSNSSDP | L | RNEWGCRCSDGSDP |
| L | RNGWECRCSNSSDP | L | RNGWGCRKSNGSDP | L | RNEWGCRCSNSSDP |
| L | RNGWECRCSNGSDP | L | RNEWECRCSDSSDP | L | RNEWGCRCSNGSDP |
| L | RNGWECRKSDSSDP | L | RNEWECRCSDGSDP | L | RNEWGCRKSDSSDP |
| L | RNGWECRKSDGSDP | L | RNEWECRCSNSSDP | L | RNEWGCRKSDGSDP |
| L | RNGWECRKSNSSDP | L | RNEWECRCSNGSDP | L | RNEWGCRKSNSSDP |
| L | RNGWECRKSNGSDP | L | RNEWECRKSDSSDP | L | RNEWGCRKSNGSDP |
| L | RNGWGCRCSDSSDP | L | RNEWECRKSDGSDP | L | RSGWECRCSDSSDP |
| L | RNGWGCRCSDGSDP | L | RNEWECRKSNSSDP | L | RSGWECRCSDGSDP |
| L | RNGWGCRCSNSSDP | L | RNEWECRKSNGSDP | L | RSGWECRCSNSSDP |
| L | RNGWGCRLSNGSDP | L | RSEWECRKSNGSDP | L | RSGWECRCSNGSDP |
| L | RSGWECRKSDSSDP | L | RSEWCRKSNGSDP | L | KNGWGCRCSNSSDP |
| L | RSGWECRKSDGSDP | L | RSEWGCRCSDSSDP | L | KNGWGCRCSNSSDP |
| L | RSGWECRKSNSSDP | L | RSEWGCRCSDGSDP | L | KNGWGCRCSNGSDP |
| L | RSGWECRKSNGSDP | L | RSEWGCRCSNSSDP | L | KNGWGCRKSDSSDP |
| L | RSGWGCRCSDSSDP | L | RSEWGCRCSNGSDP | L | KNGWGCRKSDGSDP |
| L | RSGWGCRCSDGSDP | L | RSEWGCRKSDSSDP | L | KNGWGCRKSNSSDP |
| L | RSGWGCRCSNSSDP | L | RSEWGCRKSDGSDP | L | KNGWGCRKSNGSDP |
| L | RSGWGCRCSNGSDP | L | RSEWGCRKSNSSDP | L | KNEWECRCSDSSDP |
| L | RSGWGCRKSDSSDP | L | RSEWGCRKSNGSDP | L | KNEWECRCSDGSDP |
| L | RSGWGCRKSDGSDP | L | KNGWECRCSDSSDP | L | KNEWECRCSNSSDP |
| L | RSGWGCRKSNSSDP | L | KNGWECRCSDGSDP | L | KNEWECRCSNGSDP |
| L | RSGWGCRKSNGSDP | L | KNGWECRCSNSSDP | L | KNEWECRKSDSSDP |
| L | RSEWECRCSDSSDP | L | KNGWECRCSNGSDP | L | KNEWECRKSDGSDP |
| L | RSEWECRCSDGSDP | L | KNGWECRKSDSSDP | L | KNEWECRKSNSSDP |
| L | RSEWECRCSNSSDP | L | KNGWECRKSDGSDP | L | KNEWECRKSNGSDP |
| L | RSEWECRCSNGSDP | L | KNGWECRKSNSSDP | L | KNEWGCRCSDSSDP |
| L | RSEWECRKSDSSDP | L | KNGWECRKSNGSDP | L | KNEWGCRCSDGSDP |
| L | RSEWECRKSDGSDP | L | KNGWGCRCSDSSDP | L | KNEWGCRCSNSSDP |
| L | RSEWECRKSNSSDP | L | KNGWGCRCSDGSDP | L | KNEWGCRCSNGSDP |
| L | KNEWGCRKSDGSDPL | L | KSGWGCRCSDGSDPL | L | KNEWGCRKSDSSDP |
| L | KNEWGCRKSNSSDPL | L | KSGWGCRCSNSSDPL | L | KSEWECRKSDGSDPL |
| L | KNEWGCRKSNGSDPL | L | KSGWGCRCSNGSDPL | L | KSEWECRKSNSSDPL |
| L | KSGWECRCSDSSDPL | L | KSGWGCRKSDSSDPL | L | KSEWECRKSNGSDPL |
| L | KSGWECRCSDGSDPL | L | KSGWGCRKSDGSDPL | L | KSEWGCRCSDSSDPL |
| L | KSGWECRCSNSSDPL | L | KSGWGCRKSNSSDPL | L | KSEWGCRCSDGSDPL |
| L | KSGWECRCSNGSDPL | L | KSGWGCRKSNGSDPL | L | KSEWGCRCSNSSDPL |
| L | KSGWECRKSDSSDPL | L | KSEWECRCSDSSDPL | L | KSEWGCRCSNGSDPL |
| L | KSGWECRKSDGSDPL | L | KSEWECRCSDGSDPL | L | KSEWGCRKSDSSDPL |
| L | KSGWECRKSNSSDPL | L | KSEWECRCSNSSDPL | L | KSEWGCRKSDGSDPL |
| L | KSGWECRKSNGSDPL | L | KSEWECRCSNGSDPL | L | KSEWGCRKSNSSDPL |
| L | KSGWGCRCSDSSDP | L | KSEWECRKSDSSDPL | | KSEWGCRKSNGSDPL |

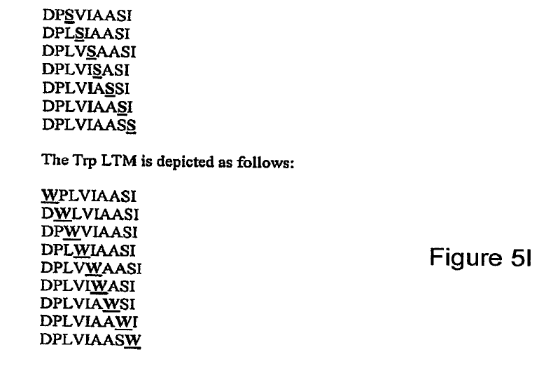

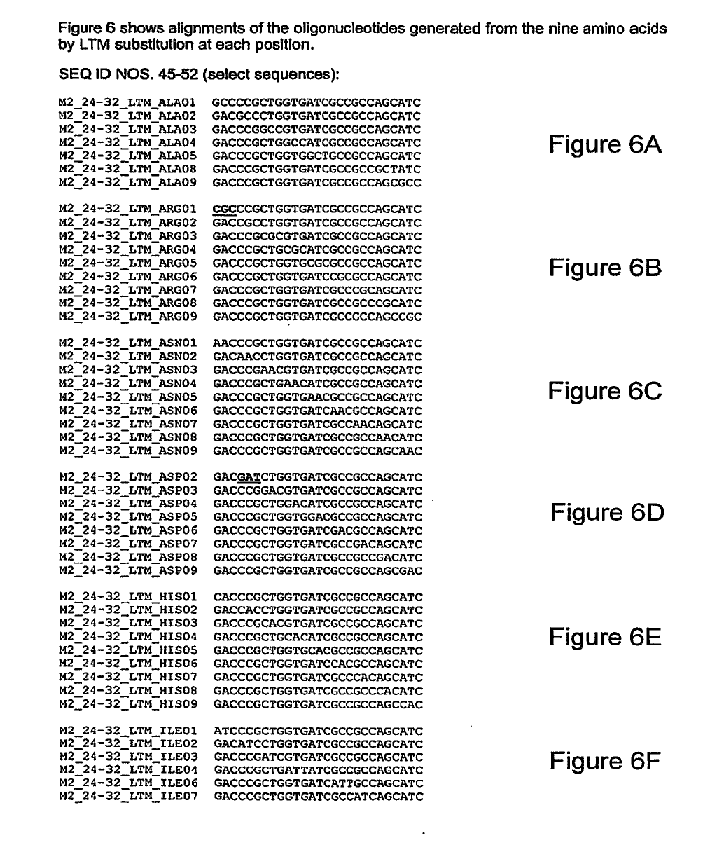

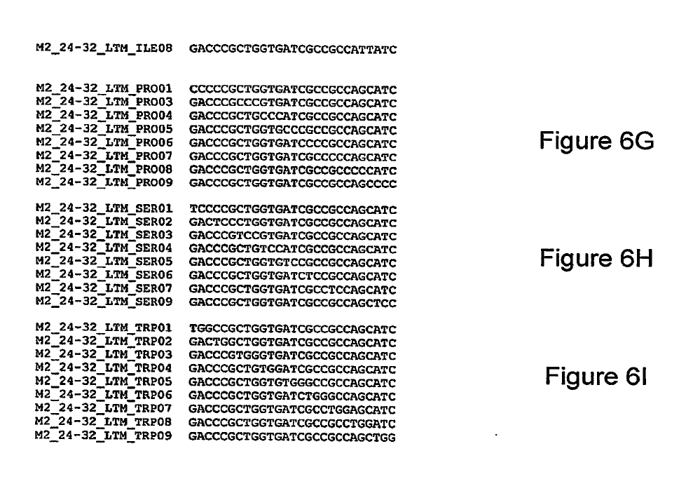

The M2 MHC II consensus RNEWECRCSDSSDPL sequence is also the starting sequence for Look Through Mutagenesis. The Look Through variants are made in the same step-wise manner as shown in FIG. 6 for the M2 MHC I binding peptide.

### M2 MHC I Binding Regions

From the same the full length M2 protein sequence query (Influenza A virus H2N3 above) of the SYFPEITHI MHC database (http://www.syfpeithi.de) we then chose the twenty highest HLA-B″ 5101 scoring peptides for further analysis (results not shown). As above we first we localized where in the M2 protein these MHC I nonamers were located.

In this case the predicted MHC I binding peptide was DPLVIAASI beginning at position 24 of the M2 protein. Examination of the M2 localization indicated that this peptide was also in an area of moderate sequence variability. In this case though, only three out of the nine positions were variable and at each position, there was only one alternative amino acid. The combinatorial possibilities for this region were much more limited as compared to the MHC II sequences above. They are as follows:

DPLVIAASI
DPLVIAANI
DPLVVAASI
DPLVVAANI
DPLTIAASI
DPLTIAANI
DPLTVAASI
DPLTVAANI

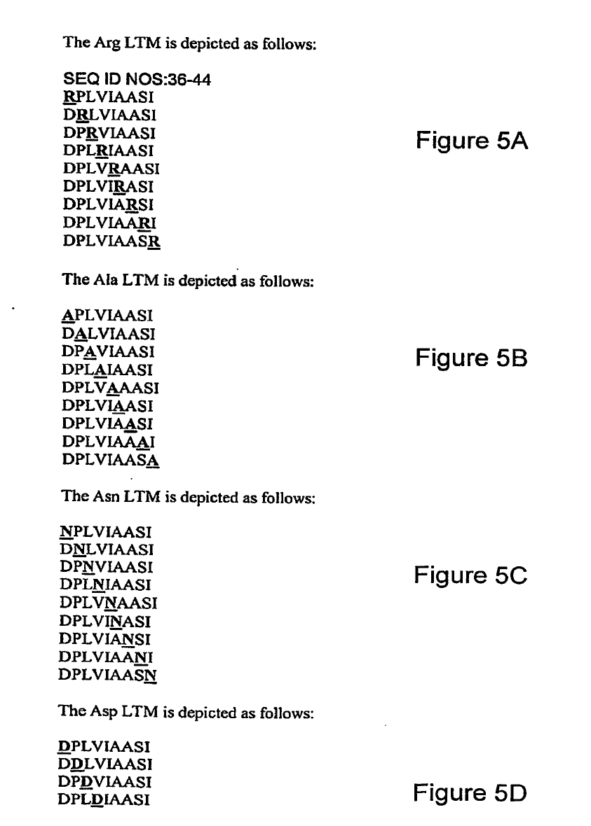

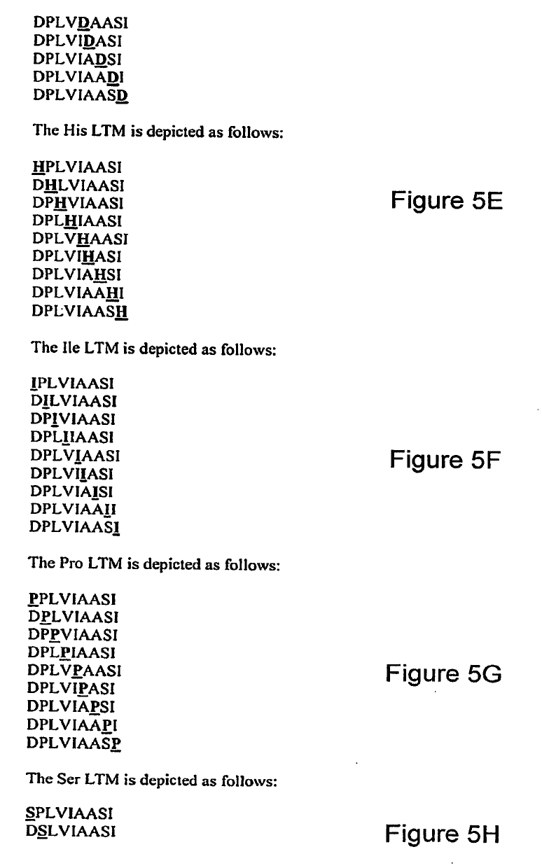

The M2 MHC I consensus DPLTVAANI sequence is also the starting sequence for Look Through Mutagenesis. The Look Through variants are made in the same step-wise manner as shown in FIG. 5 using the oligonucleotides shown in FIG. 6.

In an analogous manner as above, we queried the SYFPEITHI MHC database (http://www.syfpeithi.de) with the full length NS1 protein from Influenza A virus H3N2GenBank ID (>gi|9049379|dbj|BAA99397.1|NS1 [Influenza A virus (X-31 (H3N2))].

Here, the query sequence was:

MDPNTVSSFQVDCFLWHVRKRVADQELGDAPFLDRLRRDQKSLRGRGSTL
GLDIKTATRAGKQIVERILKEESDEALKMTMASVPASRYLTDMTLEEMSR
DWSMLKQKVAGPLCIRMDQAIMDKNIILKANFSVIFDRLETLILLRAFTE
EGAIVGEISPLPSLPGHTAEDVKNAVGVLIGGLEWNDNTVRVSETLQRFA
WRSSNENGRPPLTPKQKREMAGTIRSEV

SYFPEITHI prediction results also generated positive score for predicted binding in the available MHC alleles in the database (results not shown). In this case the MHC I binding peptide IFDRLETLI region had three positions, out of nine, that displayed variability (See FIG. 4b). These sequences then generated the following eighteen combinatorial possibilities:

IFDRLETLI
IFDRLETLT
IFDRLETLI
IFDRLEALT
IFDRLEALI
IFDRLEALT
IFGRLENLI
IFGRLENLT
IFGRLENLI
IFGRLETLT
IFGRLETLI
IFGRLETLT
IFNRLEALI
IFNRLEALT
IFNRLEALI
IFNRLENLT
IFNRLENLI
IFNRLENLT

As above, we localized the MHC II binding region to an area that exhibited a moderate amount of sequence variability. The aligned table below illustrates the consensus sequence (underlined amino acids) for the NS1 region from positions 74 to 88.

| D | E | A | L | K | M | T | I | A | S | V | P | A | S | R |
|---|---|---|---|---|---|---|---|---|---|---|---|---|---|---|
| | K | I | F | | I | N | M | T | | I | L | | P | |
| | | | | | | A | | | | A | | | | |

This table would then generate 1152 possible combinatorial peptides (all results not shown).

| | |
|---|---|
| DEALKMTIASVPASR | DEALKITIASVLASR |
| DEALKMTIASVPAPR | DEALKITIASVLAPR |
| DEALKMTIASVLASR | DEALKITITSVPASR |
| DEALKMTIASILAPR | DEALKITITSIPAPR |
| DEALKMTITSIPASR | DEALKITMTSILASR |
| DEALKMTITSIPAPR | DEALKITMTSILAPR |
| DEALKMNITSALASR | DEALKINMASAPASR |
| DEALKMNMTSALAPR | DEALKINMASAPAPR |
| DEALKMNMASAPASR | DEALKINMASALASR |
| DEALKMNMASVPAPR | DEALKINMASVLAPR |
| DEALKMNMASVLASR | DEALKINMTSVPASR |
| DEALKMNMASVLAPR | DEALKINMTSVPAPR |
| DEALKMAMTSIPASR | DEALKIAITSILASR |
| DEALKMAMTSIPAPR | DEALKMAITSILAPR |
| DEALKMAMTSILASR | DEALKMAIASIPASR |
| DEALKIAITSALAPR | DEALKMAIASAPAPR |
| DEALKIAIASAPASR | DEALKMAIASALASR |
| DEALKIAIASAPAPR | DEALKMAIASALAPR |

C. HAO, Brief Description of Applying Same Methods to HAO

The presence or absence of amino acids from an aligned sequence of a particular variant is relative to a chosen consensus length of a reference sequence, which in the example shown in FIGS. 3 and 4, were subtypes H1-H15; i.e. all fifteen subtypes were studied. In order to maintain the highest homology in alignment of sequences, deletions in the sequence of a variant relative to the reference sequence can be represented by an amino acid space “-”, while insertional mutations in the variant relative to the reference sequence can be disregarded and left out of the sequence of the variant when aligned. Given N variants of the protein, the number of times that a given amino acid (aa) occurs at a given position n, the frequency of occurrence for that amino acid at that position n is calculated, as described in co-owned U.S. Pat. No. 6,432,675, which is incorporated herein by reference. The frequency at which an amino acid deletion occurs at a given position can be factored into this calculation as well.

Alternatively, if the deletions are not considered in the frequency calculation, then it may be desirable that the value of N used in the calculation at a given amino acid position n should be the number of variants less the number of variants in which an amino acid space is present at that given position. Based upon the determination of the frequency of occurrence of amino acid types at each position n in the population of variants, a “threshold value” for inclusion of a particular amino acid type at the corresponding position n for the set of polypeptide antigens is determined. A degenerate oligonucleotide sequence can then be created. The degenerate oligonucleotide sequence is designed to have the minimum number of nucleotide combinations necessary, at each codon position, to give rise to codons for each amino acid type selected based upon the chosen threshold value, as detailed in U.S. Pat. No. 6,432,675.

The threshold frequency used to select types of amino acids for inclusion in the set of polypeptide antigens and accordingly, for determining the degenerate oligonucleotide sequence, can be applied uniformly to each amino acid position. For instance, a threshold value of 15 percent can be applied across the entire protein sequence. Alternatively, the threshold value can be set for each amino acid position n independently. For example, the threshold value can be set at each amino acid position n so as to include the most commonly occurring amino acid types, e.g., those which appear at that position in at least 90% of the N variants.

It may in some instances be desirable to apply a further criterion to the determination of a degenerate oligonucleotide sequence which comprises restricting the degeneracy of a codon position such that no more than a given number of amino acid types can arise at the corresponding amino acid position in the set of polypeptide antigens. For example, the degenerate sequence of a given codon position n can be restricted such that selected amino acids will occur in at least about 11% of the polypeptides of the polypeptide antigen set. This means that all of the possible nucleotide combinations of that degenerate codon will give rise to no more than 9 different amino acids at the position. Thus, the frequency at which a particular amino acid appears at a given position will depend on the possible degeneracy of the corresponding codon position. Preferably, the number will be 11.1 (9 different amino acids), 12.5 (8 different amino acids), 16.6 (6 different amino acids), 25 (4 different amino acids) or 50 (2 different amino acids).

Likewise, criteria used for choosing the population of variants for frequency analysis can be determined by such factors as the expected utility of the polypeptide antigen set and factors concerning vaccination or tolerization. For example, analysis of a variant protein sequence can be restricted to subpopulations of a larger population of variants of the protein based on factors such as epidemiological data, including geographic occurrence or alternatively, on known allele families (such as variants of the DQ HLA class II allele). Likewise, in the case of protein components of pathogens, the population of variants selected for analysis can be chosen based on known tropisms for a particular susceptible host organism.

Applying this approach, the amino acid variants that occur in the influenza A HA region 91-160 for the influenza A subtypes shown in FIG. 1 were each examined for frequency of occurrence above a selected threshold level. The results of this analysis are shown in FIG. 2, where a specified residue represents an invariant position and “Xaa” represents one or more possible amino acid variations at that position. In preparing the composition of this invention, polypeptides representing each of the specified variants are preferably produced. More generally, the composition includes a majority of the possible sequence variations shown, preferably at least 70%, more preferably at least 80% of the sequence variations shown.

### IV. Combinatorial Vaccines

There are many ways by which the set of polypeptide antigens can be generated from the degenerate oligonucleotide sequence. Chemical synthesis of a degenerate oligonucleotide can be carried out in an automatic DNA synthesizer, and the synthetic oligonucleotides can then be ligated into an appropriate gene for expression. A start codon (ATG) can be engineered into the sequence if desired. The degenerate oligonucleotide sequences can be incorporated into a gene construct so as to allow expression of a protein consisting essentially of the set of polypeptide antigens. Alternatively, the set of polypeptide antigens can be expressed as parts of fusion proteins. The gene library created can be brought under appropriate transcriptional control by manipulation of transcriptional regulatory sequences. It may be desirable to create fusion proteins containing a leader sequence which directs transport of the recombinant proteins along appropriate cellular secretory routes.

### V. Look-Through Mutagenesis

Look Through Mutagenesis (LTM) is used to systematically alter the wildtype sequence of each candidate polypeptide antigen. LTM systematically replaces an amino acid at each position along a given polypeptide such that no more than one mutation is made at the same time. The analysis introduces a predetermined amino acid into every position within a defined region (unless the wildtype amino acid is the same as the LTM amino acid). The purpose of LTM is to introduce a selected substitution at each target mutation position in a region of the polypeptide, e.g., the CDR regions of the variable antibody chain. Unlike combinatorial methods or walk-through mutagenesis (WTM), which allow for residue substitutions at each and every position in a single polypeptide, LTM confines substitutions to a single selected position.

In one example, arginine LTM of M2 involves serially substituting only one arginine at a time, at every residue position in the M2. FIG. 5 illustrates LTM application for introducing a arginine amino acid into each of the nine residues DPLVIAASI (positions 24-32) in the predicted MHCI a region of M2. In performing arginine LTM, nine separate oligonucleotides encoding all possible M2 arginine positional variants were synthesized with each having only one arginine replacement codon (in bold and underlined) bordered by M2 wild type sequence (indicated by xxxxx and yyyyy).

LTM oligonucleotides for the other eight “subset” amino acids; alanine, aspartate, lysine, leucine, proline, glycine, serine, tyrosine, and histidine were designed and synthesized in an analogous manner. For example, the first aspartate (codon in bold) LTM oligonucleotide replacement is in the 25th amino acid position of the M2 protein. The other aspartate LTM oligonucleotides are listed in FIG. 6.

5′- GAC GAT CTG GTG ATC GCC GCC AGC ATC -3′.

An example of oligonucleotides for asparagine LTM is listed in FIG. 6. As in the LTM design above, XX and YY base pairs of flanking wild type M2 framework allow SOE-PCR assembly into the remainder of the M2 construct.

This feature is illustrated in FIGS. 5 and 6. FIG. 5 shows the nine-residue amino acid sequence of a region of the wildtype M2 (top line) and below that, nine sequences having a single Arg substitution at each of the positions along M2. The purpose of the LTM method illustrated in FIG. 6 is to substitute a single Arg residue at each of the nine positions 24-32. This is accomplished by generating, in addition to the wildtype coding sequence, nine additional coding sequences that individually provide an kg CGT or CGC codon at each one of the nine different codon positions. A total of nine different peptides are generated, and no “undesired” or multiple-substitution sequences are produced.

Various methods of chemically synthesizing polydeoxynucleotides are known, including solid-phase synthesis which, like peptide synthesis, has been fully automated in commercially available DNA synthesizers (See the Itakura et al. U.S. Pat. No. 4,598,049; the Caruthers et al. U.S. Pat. No. 4,458,066; and the Itakura U.S. Pat. Nos. 4,401,796 and 4,373,071, incorporated by reference herein).

The purpose of a degenerate set of oligonucleotides is to provide, in one mixture, all of the sequences encoding the desired set of polypeptide antigens. It will generally not be practical to synthesize each oligonucleotide of this mixture one by one, particularly in the case of great numbers of possible variants. In these instances, the mixture can be synthesized by a strategy in which a mixture of coupling units (nucleotide monomers) are added at the appropriate positions in the sequence such that the final oligonucleotide mixture includes the sequences coding for the desired set of polypeptide antigens. Conventional techniques of DNA synthesis take advantage of protecting groups on the reactive deoxynucleotides such that, upon incorporation into a growing oligomer, further coupling to that oligomer is inhibited until a subsequent deprotecting step is provided. Thus, to create a degenerate sequence, more than one type of deoxynucleotide can be simultaneously reacted with the growing oligonucleotide during a round of coupling, either by premixing nucleotides or by programming the synthesizer to deliver appropriate volumes of nucleotide-containing reactant solutions. For each codon position corresponding to an amino acid position having only one amino acid type in the eventual set of polypeptide antigens, each oligonucleotide of the degenerate set of oligonucleotides will have an identical nucleotide sequence. At a codon position corresponding to an amino acid position at which more than one amino acid type will occur in the eventual set, the degenerate set of oligonucleotides will comprise nucleotide sequences giving rise to codons which code for those amino acid types at that position in the set. In some instances, due to other combinations that the degenerate nucleotide sequence can have, the resulting oligonucleotides will have codons directed to amino acid types other than those designed to be present based on analysis of the frequency of occurrence in the variant. The synthesis of degenerate oligonucleotides is well known in the art (see for example Narang, S A (1983) Tetrahedron 39:3; Itakura et al. (1981) in Recombinant DNA, Proc 3rd Cleveland Sympos. Macromolecules, ed. A G Walton, Amsterdam: Elsevier pp 273-289; Itakura et al. (1984) Annu. Rev. Biochem, 53:323; Itakura et al., (1984) Science 198:1056; Ike et al. (1983) Nucleic Acid Res. 11:477, incorporated by reference herein).

As noted above, one strategy of synthesizing the degenerate oligonucleotide involves simultaneously reacting more than one type of deoxynucleotide during a given round of coupling. For instance, if either a Histidine (His) or Threonine (Thr) was to appear at a given amino acid position, the synthesis of the set of oligonucleotides could be carried out as follows: (assuming synthesis were proceeding 3′ to 5′) the growing oligonucleotide would first be coupled to a 5′-protected thymidine deoxynucleotide, deprotected, then simultaneously reacted with a mixture of a 5′-protected adenine deoxynucleotide and a 5′-protected cytidine deoxynucleotide. Upon deprotection of the resulting oligonucleotides, another mixture of a 5′-protected adenine deoxynucleotide and a 5′-protected cytidine deoxynucleotide are simultaneously reacted. The resulting set of oligonucleotides will contain at that codon position either ACT (Thr), AAT (Asn), CAT (His) or CCT (Pro). Thus, when more than one nucleotide of a codon is varied, the use of nucleotide monomers in the synthesis can potentially result in a mixture of codons including, but not limited to, those designed to be present by frequency analysis.

To create an amino acid space (deletion) at a given amino acid position, a portion of the oligonucleotide mixture can be held aside during the appropriate rounds of nucleotide additions (i.e., three coupling rounds per codon) so as to lack a particular codon position all together, then added back to the mixture at the start of synthesis of the subsequent codon position.

The entire coding sequence for the polypeptide antigen set can be synthesized by this method. In some instances, it may be desirable to synthesize degenerate oligonucleotide fragments by this method, which is then ligated to invariant DNA sequences synthesized separately to create a longer degenerate oligonucleotide.

Likewise, the amino acid positions containing more than one amino acid type in the generated set of polypeptide antigens need not be contiguous in the polypeptide sequence. In some instances, it may be desirable to synthesize a number of degenerate oligonucleotide fragments, each fragment corresponding to a distinct fragment of the coding sequence for the set of polypeptide antigens. Each degenerate oligonucleotide fragment can then be enzymatically ligated to the appropriate invariant DNA sequences coding for stretches of amino acids for which only one amino acid type occurs at each position in the set of polypeptide antigens. Thus, the final degenerate coding sequence is created by fusion of both degenerate and invariant sequences.

These methods are useful when the frequency-based mutations are concentrated in portions of the polypeptide antigen to be generated and it is desirable to synthesis long invariant nucleotide sequences separately from the synthesis of degenerate nucleotide sequences.

Furthermore, the degenerate oligonucleotide can be synthesized as degenerate fragments and ligated together (i.e., complementary overhangs can be created, or blunt-end ligation can be used). It is common to synthesize overlapping fragments as complementary strands, then annealing and filling in the remaining single-stranded regions of each strand. It will generally be desirable in instances requiring annealing of complementary strands that the junction be in an area of little degeneracy.

The nucleotide sequences derived from the synthesis of a degenerate oligonucleotide sequence and encoding the set of polypeptide antigens can be used to produce the set of polypeptide antigens via microbial processes. Ligating the sequences into a gene construct, such as an expression vector, and transforming or transfecting into hosts, either eukaryotic (yeast, avian or mammalian) or prokaryotic (bacterial cells), are standard procedures used in producing other well-known proteins, e.g. insulin, interferons, human growth hormone, IL-1, IL-2, and the like. Similar procedures, or obvious modifications thereof, can be employed to prepare the set of polypeptide antigens by microbial means or tissue-culture technology in accord with the subject invention.

As stated above, the degenerate set of oligonucleotides coding for the set of polypetide antigens in the form of a library of gene constructs can be ligated into a vector suitable for expression in either prokaryotic cells, eukaryotic cells, or both. Expression vehicles for production of the set of polypeptide antigens of this invention include plasmids or other vectors. For instance, suitable vectors for the expression of the degenerate set of oligonucleotides include plasmids of the types: pBR322, pEMBL plasmids, pEX plasmids, pBTac plasmids and pUC plasmids for expression in prokaryotic cells, such as E. coli.

A number of vectors exist for the expression of recombinant proteins in yeast. For instance, YEP24, YIP5, YEP51, YEP52 and YRP17 are cloning and expression vehicles useful in the introduction of genetic constructs into S. cerevisiae (see for example Broach et al. (1983) in Experimental Manipulation of Gene Expression, ed M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eukaryotic cells. The pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. These vectors are modified with sequences from bacterial plasmids such as pBR322 to facilitate replication and drug resistance selection in both prokaryotic and eukaryotic cells. Alternatively, derivatives of viruses such as the bovine papilloma virus (BPV-1), Epstein-Barr virus (pHEBo and p205) can be used for transient expression of proteins in eukaryotic cells. The various methods employed in the preparation of the plasmids and transformation of host organisms are well known in the art. For other suitable expression systems for both prokaryotic and eukaryotic, as well as general recombinant procedures, see Molecular Cloning, 2nd Ed., ed. by Sambrook, Fritsch and Maniatis (Cold Spring Harbor Laboratory Press: 1989) incorporated by reference herein.

To express the library of gene constructs of the degenerate set of oligonucleotides, it may be desirable to include transcriptional and translational regulatory elements and other non-coding sequences to the expression construct. For instance, regulatory elements including constituitive and inducible, promoters and enhancers can be incorporated.

In some instances, it will be necessary to add a start codon (ATG) to the degenerate oligonucleotide sequence. It is well known in the art that a methionine at the N-terminal position can be enzymatically cleaved by the use of the enzyme methionine aminopeptidase (MAP). MAP has been cloned from E. coli (Ben-Bassat et al. (1987) J. Bacterial. 169:751-757) and Salmonella typhimurium and its in vitro activity has been demonstrated on recombinant proteins (Miller et al. (1987) PNAS 84:2718-1722). Therefore, removal of an N-terminal methionine if desired can be achieved either in vivo by expressing the set of polypeptide antigens in a host which produces MAP (e.g., E. coli or CM89 or S. cerevisiae) or in vitro by use of Purified MAP (e.g., procedure of Miller et al.).

Alternatively, the coding sequences for the polypeptide antigens can be incorporated as a part of a fusion gene including an endogenous protein for expression by the microorganism. For example, the VP6 capsid protein of rotavirus can be used as an immunologic carrier protein for the polypeptide antigen set, either in the monomeric form or in the form of a viral particle. The set of degenerate oligonucleotide sequences can be incorporated into a fusion gene construct which includes coding sequences for a late vaccinia virus structural protein to produce a set of recombinant viruses expressing fusion proteins comprising the set of polypeptide antigens as part of the virion. It has been demonstrated with the use of V-3 loop/Hepatitis B surface antigen fusion proteins that recombinant Hepatitis B virions can be utilized in this role as well. Similarly, chimeric constructs coding for fusion proteins containing the set of polypeptide antigens and the poliovirus capsid protein can be created to enhance immunogenecity of the set of polypeptide antigens. The use of such fusion protein expression systems to establish a set of polypeptide antigens has the advantage that often both B-cell proliferation in response to the immunogen can be elicited. (see for example EP Publication No. 0259149; and Evans et al. (1989) Nature 339:385; Huang et al. (1988) J. Virol. 62:3855; and Schlienger et al. (1992) J. Virol. 66:2, incorporated by reference herein). The Multiple Antigen Peptide (MAP) system for peptide-based vaccines can be utilized in which the polypeptide antigen set is obtained directly from organo-chemical synthesis of the peptides onto an oligomeric branching lysine core (see for example Posnett et al. (1988) JBC 263:1719 and Nardelli et al. (1992) J. Immunol. 148:914, incorporated by reference herein). Foreign antigenic determinants can also be expressed and presented by bacterial cells.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. Alternatively, the fusion gene can be synthesized by conventional techniques including automated DNA synthesizers.

An alternative approach to generating the set of polypeptide antigens is to carry out the peptide synthesis directly. At each codon position n in the degenerate oligonucleotide, each possible nucleotide combination can be determined and the corresponding amino acid designated for inclusion at the corresponding amino acid position of the polypeptide antigen set. Thus, synthesis of a degenerate polypeptide sequence can be directed in which sequence divergence occurs at those amino acid positions at which more than one amino acid is coded for in the corresponding codon position of the degenerate oligonucleotide. Organo-chemical synthesis of polypeptides is well known and can be carried out by procedures such as solid state peptide synthesis using automated protein synthesizers.

The synthesis of polypeptides is generally carried out through the Condensation of the carboxy group of an amino acid, and the amino group of another amino acid, to form a peptide bond. A sequence can be constructed by repeating the condensation of individual amino acid residues in stepwise elongation, in a manner analogous to the synthesis of oligonucleotides. In such condensations, the amino and carboxy groups that are not to participate in the reaction can be blocked with protecting groups which are readily introduced, stable to the condensation reactions and selectively removable from the completed peptide. Thus, the overall process generally comprises protection, activation, coupling and deprotection. If a peptide involves amino acids with side chains that may react during condensation, the side chains can also be reversibly protected, removable at the final stage of synthesis.

A successful synthesis for a large polypeptide by a linear strategy must achieve nearly quantitative recoveries for each chemical step. Many automated peptide synthesis schemes take advantage of attachment of the growing polypeptide chain to an insoluble polymer resin support such that the polypeptide can be washed free of byproducts and excess reactants after each reaction step (see for example Merrifield (1963) J.A.C.S. 85:2149; Chang et al. (1978) Int. J. Peptide Protein Res. 11:246; Barany and Merrifield, The Peptides, vol 2.COPYRGT.1979 NY:Academic Press, pp 1-284; Tam, J. P. (1988) PNAS 85:5409; and Tam et al. U.S. Pat. No. 4,507,230, incorporated by reference herein). For example, a first amino acid is attached to a resin by a cleavable linkage to its carboxylic group, deblocked at its amino acid side, and coupled with a second activated amino acid carrying a protected .alpha.-amino group. The resulting protected dipeptide is deblocked to yield a free amino terminus, and coupled to a third N-protected amino acid. After many repetitions of these steps, the complete polypeptide is cleaved from the resin support and appropriately deprotected.

To generate the set of polypeptide antigens, more than one N-protected amino acid type can be reacted simultaneously in each round of coupling with the growing polypeptide chain to create the desired degenerate amino acid sequence at each amino acid position. In one embodiment, the set of polypeptides will include only those amino acids that are present at any position n in the population of variants above the predetermined threshold frequency.

Alternatively, one can first design the degenerate oligonucleotide, determine the amino acids encoded by the combination of codons and include all the amino acids in the chemical synthesis. For example, a degenerate codon at codon position n, having the sequence MMT and thus coding for either a Thr (ACT), an Asn (AAT), a His (CAT) or a Pro (CCT), can be created at the peptide synthesis level by reacting all four N-protected amino acid types simultaneously with the free amino terminus of the growing, resin-bound peptide. Thus, four subpopulations of peptides will be created, each subpopulation definable by the amino acid type present at the amino acid position n corresponding to the codon position n.

Because the amino acid being added to the resin-bound polypeptide is protected, the growth of the peptide chain is terminated upon addition of the protected amino acid until the subsequent deblocking step. Those skilled in the art will recognize that, due to potential differences in reactivity of various amino acid analogs, it may be desirable to use non-equimolar ratios of amino acid types when simultaneously reacting more than one amino acid type in order to get equimolar ratios of subpopulations. Alternatively, it may be desirable to divide the resin-bound polypeptide into aliquots, each of which is reacted with a distinct amino acid type, the polypeptide products being recombined prior to the next coupling reaction. This technique can be applied to create an amino acid gap in a subpopulation, simply by holding aside an appropriate aliquot during one round of coupling, then recombining all resin-bound polypeptides prior to the next round of coupling. Furthermore, it is apparent that, from the many different blocking and activating groups available, chemical synthesis of the polypeptide can be carried out in either the N-terminal to C-terminal, or C-terminal to N-terminal direction.

The generated set of polypeptide antigens can be covalently or noncovalently modified with non-proteinaceous materials such as lipids or carbohydrates to enhance immunogenecity or solubility. The present invention is understood to include all such chemical modifications of the set of polypeptide antigens so long as the modified peptide antigens retain substantially all the antigenic/immunogenic properties of the parent mixture.

The generated set of polypeptide antigens can also be coupled with or incorporated into a viral particle, a replicating virus, or other microorganism in order to enhance immunogenicity. The set of polypeptide antigens may be chemically attached to the viral particle or microorganism or an immunogenic portion thereof.

There are a large number of chemical cross-linking agents that are known to those skilled in the art. For the present invention, the preferred cross-linking agents are heterobifunctional cross-linkers, which can be used to link proteins in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art. These include: succinimidyl 4-(N-maleimidomethyl)cyclohexane-1-carboxylate (SMCC), m-Maleimidobenzoyl-N-hydroxysuccinimide ester (MBS); N-succinimidyl (4-iodoacetyl)aminobenzoate (SIAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-.alpha.-methyl-.alpha.-(2-pyridyldithio)-toluene (SMPT), N-succinimidyl 3-(2-pyridyldithio)propionate (SPDP), succinimidyl 6-[3-(2-pyridyldithio)propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

The introduction of antigen into an animal initiates a series of events culminating in both cellular and humoral immunity. By convention, the property of a molecule that allows it to induce an immune response is called immunogenicity. The property of being able to react with an antibody that has been induced is called antigenicity. Antibodies able to cross-react with two or more different antigens can do so by virtue of some degree of structural and chemical similarity between the antigenic determinants (or “epitopes”) of the antigens. A protein immunogen is usually composed of a number of antigenic determinants. Hence, immunizing with a protein results in the formation of antibody molecules with different specificities, the number of different antibodies depending on the number of antigenic determinants and their inherent immunogenicity.

Proteins are highly immunogenic when injected into an animal for whom they are not normal (“self”) constituents. Conversely, peptides and other compounds with molecular weights below about 5000 (termed “haptens”) daltons, by themselves, do not generally elicit the formation of antibodies. However, if these small molecule antigens are first coupled with a longer immunogenic antigen such as a protein, antibodies can be raised which specifically bind epitopes on the small molecules. Conjugation of haptens to carrier proteins can be carried out as described above.

When necessary, modification of such ligand to prepare an immunogen should take into account the effect on the structural specificity of the antibody. That is, in choosing a site on a ligand for conjugation to a carrier such as protein, the selected site is chosen so that administration of the resulting immunogen will provide antibodies which will recognize the original ligand. Furthermore, not only must the antibody recognize the original ligand, but significant characteristics of the ligand portion of the immunogen must remain so that the antibody produced after administration of the immunogen will more likely distinguish compounds closely related to the ligand which may also be present in the patient sample. In addition, the antibodies should have high binding constants.

Vaccines comprising the generated set of polypeptide antigens, and variants thereof having antigenic properties, can be prepared by procedures well known in the art. For example, such vaccines can be prepared as injectables, e.g., liquid solutions or suspensions. Solid forms for solution in, or suspension in, a liquid prior to injection also can be prepared. Optionally, the preparation also can be emulsified. The active antigenic ingredient or ingredients can be mixed with excipients which are pharmaceutically acceptable and compatible with the active ingredient. Examples of suitable excipients are water, saline, dextrose, glycerol, ethanol, or the like, and combinations thereof. In addition, if desired, the vaccine can contain minor amounts of auxiliary substances such as wetting or emulsifying agents, pH buffering agents, or adjuvants such as aluminum hydroxide or muramyl dipeptide or variations thereof. In the case of peptides, coupling to larger molecules such as Keyhole limpet hemacyanin (KLH) sometimes enhances immunogenicity. The vaccines are conventionally administered parenterally, by injection, for example, either subcutaneously or intramuscularly. Additional formulations which are suitable for other modes of administration include suppositories and, in some cases, oral formulations. For suppositories, the traditional binders and carriers include, for example, polyalkalene glycols or triglycerides. Suppositories can be formed from mixtures containing the active ingredient in the range of about 0.5% to about 10%, preferably about 1% to about 2%. Oral formulations can include such normally employed excipients as, for example, pharmaceutical grades of mannitol, lactose, starch, magnesium stearate, sodium saccharine, cellulose, magnesium carbonate, and the like. These compositions can take the form of solutions, suspensions, tablets, pills, capsules, sustained release formulations or powders and contain from about 10% to about 95% of active ingredient, preferably from about 25% to about 70%.

The active compounds can be formulated into the vaccine as neutral or salt forms. Pharmaceutically acceptable salts include the acid addition salts (formed with the free amino groups of the polypeptides) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Viruses contain many molecules that are distinguished as being foreign to the body. Their antigens, or epitopes are specifically recognized by the B cell and T cell receptors and results in cellular activation. Each individual T cell or B cell will only recognize and respond to its individual cognate “epitope”. Once activated, activated CD8+ CTL T cells will attack and destroy cells infected by the invading virus. Other CD4+ T cell (Th) or B cell may respond by making many duplicate copies of itself and remain in the body as memory cells. If the body is re-invaded by the virus in the future, these memory cells will be reactivated and respond faster and more powerfully to destroy the virus. This is the principle behind vaccines, such as the vaccinations we received in childhood against measles or mumps.

T cells recognize epitopes displayed in the context of major histocompatibility complexes (MHC, also known as HLA for Human Leoocyte antigens) via their T cell receptors. The division of CD8+ T cells and CD4+ T cells also engage different type MHC complexes. CD8+ T cells recognize epitopes in the context of MHC class I molecules, whereas CD4+ T cells recognize peptide-antigens in the context of MHC class II. As stated above, CD4+ and CD8+ T cells differ in their immune responses. CD4+ T mediated is more complex, by providing help via cytokine production to other immune system components namely, B-cells and/or CD84+ T cells. On the other hand, CD8+ T cell is simpler as these CTLs directly destroy cells expressing MHC class I complexes with the foreign epitope. Therefore, cytotoxic CD8+ T lymphocytes (CTL)-mediated immune responses play a central role in protective immunity against many viral and intracellular bacterial infections.

Another important factor include the ability of the cellular antigen processing machinery to generate a certain peptide-MHC complex by the antigen presenting cell (APC). Many molecules have been identified that participate in the process of antigen presentation including the proteasome, a multicatalytic protease and TAP (transporters associated with antigen processing) molecules. Antigen processing events appear to have peptide-dependent activity which bias certain amino acid residues and sequences for presentation on MHC I and MHC II. Therefore is it important to identify binding epitopes that elicit T-cell responses in humans. Some assays to test T-cell responses after in vitro stimulation include: cytotoxicity assays, proliferation assays, cytokine measurements, flow cytometry analyses.

A vaccine composition may include peptides containing a cocktail of multipeptide CD8 T and CD4 T helper cell focused epitopes in combination with protein fragments containing the principal neutralizing domain. For instance, several of these epitopes have been mapped within the HIV envelope, and these regions have been shown to stimulate proliferation and lymphokine release from lymphocytes. Providing both of these epitopes in a vaccine comprising a generated set of polypeptide antigens derived from analysis of HIV-1 isolates can result in the stimulation of both the humoral and the cellular immune responses. In addition, commercial carriers and adjuvants are available to enhance immunomodulation of both B-cell and T-cell populations for an immunogen (for example, the IMJECT SUPERCARRIER™ System, Pierce Chemical, Catalog No. 77151G).

Alternatively, a vaccine composition may include a compound which functions to increase the general immune response. One such compound is interleukin-2 (IL-2) which has been reported to enhance immunogenicity by general immune stimulation (Nunberg et al. (1988) In New Chemical and Genetic Approaches to Vaccination, Cold Spring Harbor Laboratory, Cold Spring Harbor, N.Y.). IL-2 may be coupled the polypeptides of the generated set of polypeptide antigens to enhance the efficacy of vaccination.

The vaccines are administered in a manner compatible with the dosage formulation, and in such amount as will be therapeutically effective and immunogenic. The quantity to be administered depends on the subject to be treated, capacity of the subjects immune system to synthesize antibodies, and the degree of protection desired. Precise amounts of active ingredient required to be administered depend on the judgment of the practitioner and are peculiar to each individual. Suitable regimes for initial administration and booster shots are also variable, but are typified by an initial administration followed in one or two week intervals by a subsequent injection or other administration.

Antigens that induce tolerance are called toleragens, to be distinguished from immunogens, which generate immunity. Exposure of an individual to immunogenic antigens stimulates specific immunity, and for most immunogenic proteins, subsequent exposures generate enhanced Secondary responses. In contrast, exposure to a toleragenic antigen not only fails to induce specific immunity, but also inhibits lymphocyte activation by subsequent administration of immunogenic forms of the same antigen. Many foreign antigens can be immunogens or toleragens, depending on the physicochemical form, dose, and route of administration. This ability to manipulate responses to antigens can be exploited clinically to augment or suppress specific immunity. For instance, it can be desirable in the context of organ transplant technology to tolerize a transplant recipient with a polypeptide antigen set derived from the frequency analysis of known sub-haplotypes of a class II peptide (i.e., such as the DQ or DR allele products) present on the transplanted tissue in order to minimize rejection. It is also within the equivalence of this invention that the set of polypeptide antigens can be chemically coupled or incorporated as part of a fusion protein with an apoptotic agent, for instance an agent which brings about deregulation of C-myc expression or a cell toxin such as diptheria toxoid, such that programmed cell death is brought about in an antigen specific manner.

Thus it would be routine for one skilled in the art to determine the appropriate administration regimen necessary to induce tolerance to the set of polypeptide antigens of the present invention.

### EXAMPLES

### Example 1

The following example illustrates the methodology for identifying candidate Influenza regions for the peptide vaccine. We queried the SYFPEITHI MHC database (http://www.syfpeithi.de) with the full length M2 protein from Influenza A virus H2N3 (Genbank ID: gi|13182926|gb|AAK14988.1|AF231361_{—}1). The query sequence was:

MSLLTEVETPTRNGWECKCSDSSDPLVIAASIIGILHLILWILDRLFFKC
IYRRLKYGLKRGPSTEGVPESMREEYRQEQQSAVDVDDGHFVSIELE

### M2 MHC II Candidate Peptides

SYFPEITHI prediction results demonstrated that some of the modeled HLA alleles gave a positive score for predicted binding. An example output format is presented below for the HLA-DRB1*0101 allele.

[symbol]

| Pos | [symbol] | score |
|---|---|---|
| 37 | MLILWILDRLFFKCI | 27 |
| 40 | LWILDRLFFHCIYRR | 25 |
| 12 | RMEWECRCSDSSDPL | 24 |
| 32 | IIGILHLILWILDRL | 24 |
| [symbol] | [symbol] | 24 |
| 21 | [symbol] | 23 |
| 36 | LHLILHILDRLFFKC | 23 |
| 1 | [symbol] | 22 |
| 22 | [symbol] | 22 |
| 29 | AASIIGILHLILWXL | 22 |
| 75 | EYRQEQQSAVDVDDG | 21 |
| 44 | DRLFFKCIYRRLKYG | 20 |
| 49 | KCIYRALKYGLKRGP | 20 |
| 52 | YRRLKYGLKRGPSYA | 20 |
| 73 | [symbol] | 19 |
| 24 | [symbol] | 19 |
| 30 | ASIIGILHLILWILD | 19 |
| 54 | [symbol] | 18 |
| 27 | [symbol] | 18 |
| 45 | RLFFKCIYARLKYGL | 18 |
| 45 | [symbol] | 18 |
| 57 | YGLNRGPSVAGVPES | 17 |
| 35 | [symbol] | 17 |
| 55 | LKYALKRGPSTAGVP | 16 |
| 14 | [symbol] | 16 |
| 20 | [symbol] | [symbol] |
| 25 | [symbol] | [symbol] |
| 53 | RRLKYGLKRGPSTAG | [symbol] |
| 56 | KYGLKRGPSTAGVPE | [symbol] |
| 55 | GLKRGPSTAGVPESM | [symbol] |
| 83 | [symbol] | [symbol] |

[symbol]

indicates data missing or illegible when filed

The left hand column indicates the relative amino acid position of the presented peptide in the M2 sequence. The middle column is the 15-mer peptide sequences postulated to bind HLA-DRB1*0101 MHC II beginning at top with the fittest binder. The right column is the SYFPEITHI score for that particular peptide sequence. For example, the sequence “H L I L W I L D R L F F K C I” beginning at position 37 returned a SYFPEITHI prediction score of 27.

We then examined all MHC II binding peptides with a score greater than 20 and determined their location in the M2 full length. The above peptide sequence “HLILWILDRLFFKCI” was not found in a region of moderate sequence variability (See FIG. 3a). The next highest scoring peptide beginning at position 40 LWILDRLFFKCIYRR” returning a SYFPEITHI score of 25 was also found to be in a region of high sequence conservation. However, the third peptide that returned a SYFPEITHI score of 24 was “RNEWECRCSDSSDPL” and began at position 12 which as within the M2 variable region. The aligned isolate H1-H15 M2 sequences for this region illustrated that there were a moderate amount of amino acid changes between isolates from positions 12 to 26. The aligned table below illustrates the consensus sequence (underlined amino acids) for the M2 region. Below each consensus amino acid is the amino acid variability for that M2 position as exhibited by the other fifteen examined strain.

| | | | | | | | | | | | | | | | | |
|---|---|---|---|---|---|---|---|---|---|---|---|---|---|---|---|---|
| 12 | R | N | G | W | E | C | R | C | S | D | S | S | D | P | L | 26 |

| | | | | | | |
|---|---|---|---|---|---|---|
| K | S | E | G | K | N | G |

The combinatorial peptide that could be generated from this section is illustrated in FIG. 3c and listed as SEQ ID NO:17 where a specified residue represents an invariant position and “Xaa” represents one or more possible amino acid variations at that position.

This positional sequence variability then directs the possible M2₁₂₋₂₆ combinatorial variants derived from the RNEWECRCSDSSDPL MHC II peptide. These seven epitope positions that have two variations (2⁷) yield a total of 128 different combinatorial possibilities and are listed below:

RNGWECRCSDSSDPL
RNGWECRCSDGSDPL
RNGWECRCSNSSDPL
RNGWECRCSNGSDPL
RNGWECRKSDSSDPL
RNGWECRKSDGSDPL
RNGWECRKSNSSDPL
RNGWECRKSNGSDPL
RNGWGCRCSDSSDPL
RNGWGCRCSDGSDPL
RNGWGCRCSNSSDPL
RNGWGCRCSNGSDPL
RNGWGCRKSDSSDPL
RNGWGCRKSDGSDPL
RNGWGCRKSNSSDPL
RNGWGCRKSNGSDPL
RNEWECRCSDSSDPL
RNEWECRCSDGSDPL
RNEWECRCSNSSDPL
RNEWECRCSNGSDPL
RNEWECRKSDSSDPL
RNEWECRKSDGSDPL
RNEWECRKSNSSDPL
RNEWECRKSNGSDPL
RNEWGCRCSDSSDPL
RNEWGCRCSDGSDPL
RNEWGCRCSNSSDPL
RNEWGCRCSNGSDPL
RNEWGCRKSDSSDPL
RNEWGCRKSDGSDPL
RNEWGCRKSNSSDPL
RNEWGCRKSNGSDPL
RSGWECRCSDSSDPL
RSGWECRCSDGSDPL
RSGWECRCSNSSDPL
RSGWECRCSNGSDPL
RSGWECRKSDSSDPL
RSGWECRKSDGSDPL
RSGWECRKSNSSDPL
RSGWECRKSNGSDPL
RSGWGCRCSDSSDPL
RSGWGCRCSDGSDPL
RSGWGCRCSNSSDPL
RSGWGCRCSNGSDPL
RSGWGCRKSDSSDPL
RSGWGCRKSDGSDPL
RSGWGCRKSNSSDPL
RSGWGCRKSNGSDPL
RSEWECRCSDSSDPL
RSEWECRCSDGSDPL
RSEWECRCSNSSDPL
RSEWECRCSNGSDPL
RSEWECRKSDSSDPL
RSEWECRKSDGSDPL
RSEWECRKSNSSDPL
RSEWECRKSNGSDPL
RSEWGCRCSDSSDPL
RSEWGCRCSDGSDPL
RSEWGCRCSNSSDPL
RSEWGCRCSNGSDPL
RSEWGCRKSDSSDPL
RSEWGCRKSDGSDPL
RSEWGCRKSNSSDPL
RSEWGCRKSNGSDPL
KNGWECRCSDSSDPL
KNGWECRCSDGSDPL
KNGWECRCSNSSDPL
KNGWECRCSNGSDPL
KNGWECRKSDSSDPL
KNGWECRKSDGSDPL
KNGWECRKSNSSDPL
KNGWECRKSNGSDPL
KNGWGCRCSDSSDPL
KNGWGCRCSDGSDPL
KNGWGCRCSNSSDPL
KNGWGCRCSNGSDPL
KNGWGCRKSDSSDPL
KNGWGCRKSDGSDPL
KNGWGCRKSNSSDPL
KNGWGCRKSNGSDPL
KNEWECRCSDSSDPL
KNEWECRCSDGSDPL
KNEWECRCSNSSDPL
KNEWECRCSNGSDPL
KNEWECRKSDSSDPL
KNEWECRKSDGSDPL
KNEWECRKSNSSDPL
KNEWECRKSNGSDPL
KNEWGCRCSDSSDPL
KNEWGCRCSDGSDPL
KNEWGCRCSNSSDPL
KNEWGCRCSNGSDPL
KNEWGCRKSDSSDPL
KNEWGCRKSDGSDPL
KNEWGCRKSNSSDPL
KNEWGCRKSNGSDPL
KSGWECRCSDSSDPL
KSGWECRCSDGSDPL
KSGWECRCSNSSDPL
KSGWECRCSNGSDPL
KSGWECRKSDSSDPL
KSGWECRKSDGSDPL
KSGWECRKSNSSDPL
KSGWECRKSNGSDPL
KSGWGCRCSDSSDPL
KSGWGCRCSDGSDPL
KSGWGCRCSNSSDPL
KSGWGCRCSNGSDPL
KSGWGCRKSDSSDPL
KSGWGCRKSDGSDPL
KSGWGCRKSNSSDPL
KSGWGCRKSNGSDPL
KSEWECRCSDSSDPL
KSEWECRCSDGSDPL
KSEWECRCSNSSDPL
KSEWECRCSNGSDPL
KSEWECRKSDSSDPL
KSEWECRKSDGSDPL
KSEWECRKSNSSDPL
KSEWECRKSNGSDPL
KSEWGCRCSDSSDPL
KSEWGCRCSDGSDPL
KSEWGCRCSNSSDPL
KSEWGCRCSNGSDPL
KSEWGCRKSDSSDPL
KSEWGCRKSDGSDPL
KSEWGCRKSNSSDPL
KSEWGCRKSNGSDPL

### M2 MHC I Candidate Peptides

The SYPEITHI epitope prediction analysis described above in the Example 1 also identified a MHC I epitope for the HLA-B*5101 allele. This HLA-B*5101 epitope is shown with respect to FIG. 3B and comprises the M2₂₅₋₃₃ consensus sequence DPLVIAASI from positions 25-33. Investigation of the M2₂₅₋₃₃ region shows minor amino acid substitutions (2 possible amino acids at each variable position) in three out of nine positions. Suitable combinatorial mutagenesis can be achieved as this MHC I region contains a plurality (2 or more) of residue positions at which a small number of amino acid variations occur.

The combinatorial peptide that could be generated from this section is illustrated in FIG. 3c and listed as SEQ ID NO:16 where a specified residue represents an invariant position and “Xaa” represents one or more possible amino acid variations at that position. The MHC I combinatorial possibilities for this M2₂₅₋₃₃ region are more limited as compared to the MHC II M2₁₂₋₂₅ sequences above. They are as follows:

| | | | | | | | | | | |
|---|---|---|---|---|---|---|---|---|---|---|
| 25 | D | P | L | V | I | A | A | S | I | 33 |

| | | |
|---|---|---|
| T | V | N |

DPLVIAASI
DPLVIAANI
DPLVVAASI
DPLVVAANI
DPLTIAASI
DPLTIAANI
DPLTVAASI
DPLTVAANI

These combinatorial variants were then re-queried in the SYFPEITHI algorithm to determine the resultant score for fitness of MHC I binding. Each of the combinatorial variants yielded SYFPEITHI scores no worse than the wild type (WT) sequence and are listed as SEQ ID NOS: 58-63.

| Sequence: | SYFPEITHI score |
|---|---|
| DPLVIAASI (WT) | 27 |
| DPLTIAASI | 28 |
| DPLVVAASI | 29 |
| DPLVIAANI | 27 |
| DPLIVAASI | 30 |
| DPLTIAANI | 30 |
| DPLVVAANI | 29 |
| DPLTVAANI | 30 |

### Improvement of M2 MHC I Candidate Peptides by Look Through Mutagenesis

The HLA-B*5101 M2₂₅₋₃₃ consensus DPLTVAANI epitope sequence is also the starting sequence for Look Through Mutagenesis as described in co-owned U.S. Patent Application No. 20050136428 which is incorporated herein by reference. The LTM variants are made in the same step-wise manner as shown in FIG. 5 using the oligonucleotides shown in FIG. 6. Unlike the Combinatorial Method, LTM confines substitutions to a single selected position. Typically in the LTM method, a subset of natural amino acids, e.g., nine different amino acids that represent the physico-chemical spectrum of properties of natural amino acids, are selected. The LTM variants are made in the same step-wise manner as shown in FIG. 6 for the M2₂₅₋₃₃ MHC I binding peptides. Look Through Mutagenesis on the wild type DPLVIAASI peptide which had a SYFPEITHI score of 27. The new LTM variants were then re-queried in the SYFPEITHI algorithm to again determine the resultant score for fitness of binding. A few of the M2₂₅₋₃₃ LTM variants yield higher prediction scores compared to the wild type and correspond to SEQ ID NOS: 53-57.

| Sequence: | | SYFPEITHI score |
|---|---|---|
| DPLVIAASI | (WT) | 27 |
| DPLVIAARI | | 28 |
| DPLVIAASR | | 28 |
| DPLVSAASI | | 28 |
| DPLVIAAYI | | 29 |
| DPLVIAASY | | 28 |

### Generation of Combinatorial Peptides for NS1 MHC Binding Epitopes

In an analogous manner as above, the SYFPEITHI MHC database (http://www.syfpeithi.de) was queried with the full length NS1 protein from Influenza A virus H3N2GenBank ID (>gi|9049379|dbj|BAA99397.1|NS1 [Influenza A virus (X-31(H3N2))] using the sequence:

MDPNTVSSFQVDCFLWHVRKRVADQELGDAPFLDRLRRDQKSLRGRGSTL
GLDIKTATRAGKQIVERILKEESDEALKMTMASVPASRYLTDMTLEEMSR
DWSMLKQKVAGPLCIRMDQAIMDKNIILKANFSVIFDRLETLILLRAFTE
EGAIVGEISPLPSLPGHTAEDVKNAVGVLIGGLEWNDNWRVSETLQRFAW
RSSNENGRPPLTPKQKREMAGTIRSEV

The SYFPEITHI program generated prediction scores for various NS1 epitope regions in all available MHC alleles within the database (results not shown). In this case, one of the MHC I binding peptide sequences was IFDRLETLI from positions 137 to 145 and specific for the HLAB*5101 allele. The NS1₁₃₇₋₁₄₅ region had three positions, out of nine, that displayed variability (See FIG. 4b). In this case though, only three out of the nine positions were variable and, at each position, there was only one alternative amino acid (see table below). Inspection of position 139 shows D, G, and N are the variable amino acids, position 143 is variable for T, A and N, and position 145 is variable for 1 and T.

| 137 | I | F | D | R | L | E | T | L | I | 145 |
|---|---|---|---|---|---|---|---|---|---|---|
| | | | G | | | | A | | T | |
| | | | N | | | | N | | | |

The combinatorial peptide that could be generated from this section is listed SEQ ID NO 91: where a specified residue represents an invariant position and “Xaa” represents one or more possible amino acid variations at that position.

These three variable sequences then generated the following eighteen combinatorial possibilities:

IFDRLETLI
IFDRLETLT
IFDRLETLI
IFDRLEALT
IFDRLEALI
IFDRLEALT
IFGRLENLI

IFGRLENLT
IFGRLENLI
IFGRLETLT
IFGRLETLI
IFGRLETLT
IFNRLEALI
IFNRLEALT
IFNRLEALI
IFNRLENLT
IFNRLENLI
IFNRLENLT

In performing similar analysis for the MHC II HLA-DRB1*0101 allele, we localized the MHC II binding region to protein region that exhibited a moderate amount of sequence variability. The aligned table below illustrates the consensus sequence (underlined amino acids) for the NS1 region from positions 74 to 88.

| | | | | | | | | | | | | | | | | |
|---|---|---|---|---|---|---|---|---|---|---|---|---|---|---|---|---|
| 74 | D | E | A | L | K | M | T | I | A | S | V | P | A | S | R | 88 |

| | | | | | | | | | |
|---|---|---|---|---|---|---|---|---|---|
| K | I | F | I | N | M | T | I | L | P |
| | | | | A | | | A | | |

The combinatorial peptide that could be generated from this section is listed SEQ ID NO 92: where a specified residue represents an invariant position and “Xaa” represents one or more possible amino acid variations at that position.

This table would then generate 1152 possible combinatorial peptides (and as result only a representative sample of the results are shown below).

DEALKMTIASVPASR
DEALKMTIASVPAPR
DEALKMTIASVLASR
DEALKMTIASILAPR
DEALKMTITSIPASR
DEALKMTITSIPAPR

DEALKMNITSALASR
DEALKMNMTSALAPR
DEALKMNMASAPASR
DEALKMNMASVPAPR
DEALKMNMASVLASR
DEALKMNMASVLAPR
DEALKMAMTSIPASR
DEALKMAMTSIPAPR
DEALKMAMTSILASR
DEALKIAITSALAPR
DEALKIAIASAPASR
DEALKIAIASAPAPR
DEALKITIASVLASR
DEALKITIASVLAPR
DEALKITITSVPASR
DEALKITITSIPAPR
DEALKITMTSILASR
DEALKITMTSILAPR
DEALKINMASAPASR
DEALKINMASAPAPR
DEALKINMASALASR
DEALKINMASVLAPR
DEALKINMTSVPASR
DEALKINMTSVPAPR
DEALKIAITSILASR
DEALKMAITSILAPR
DEALKMAIASIPASR
DEALKMAIASAPAPR
DEALKMAIASALASR
DEALKMAIASALAPR

### Example 2

### LTM and CM Optimization of Known Influenza MHC Epitopes

As an alternative to the use of MHC computer predictive models, peptides that are naturally processed and presented in the context of human (MHC) HLA-A*0201 allele can be chosen as candidate epitopes. A similar direct approach has been used to identify tumor associated antigens (TAAs) whereby in vivo bound peptides are eluted from HLA molecules and then characterized by mass spectrometry [Hunt et al, 1992, Science. 255: 1261]. Data-mining of the LANL Flubase revealed that the NS1 peptide fragment (positions 122-130) was presented by “humanized” HLA-A2.1 transgenic mice. This study recovered two overlapping NS1 peptides, a nonamer AIMDKNIIL and a decamer IMDKNIILKA demonstrating the specific HLA-A2.1 preference for this particular epitope. Data-mining also catalogued the M1 protein epitope comprising the GILGFVFTL (positions 58-66) sequence. For the M2 58-66 epitope GILGFVFTL there was near total sequence conservation in after alignment of all Influenza M1 protein sequences.

Table 1 (column 1) illustrates Arg-LTM application in serially substituting one R into each of the nine residues M2 58-66 epitope GILGFVFTL in HLA-A*0201. In performing LTM, all possible M2 positional oligonucleotide variants are synthesized with each having only one corresponding LTM replacement codon (in bold and underlined). Table I also illustrates LTM replacements for Asp (D), Lys (K), Pro (P), and Gly (G) and are designed in an analogous manner (The LTM series for Leu, Ser, Lyr, and His replacements are not shown but are analogous).

TABLE 1

| R LTM | D LTM | K LTM | P LTM | G LTM |
|---|---|---|---|---|
| RILGFVFTL | DILGFVFTL | KILGFVFTL | PILGFVFTL | GILGFVFTL |
| GRLGFVFTL | GDLGFVFTL | GKLGFVFTL | GPLGFVFTL | GGLGFVFTL |
| GIRGFVFTL | GIDGFVFTL | GIKGFVFTL | GIPGFVFTL | GIGGFVFTL |
| GILRFVFTL | GILDFVFTL | GILKFVFTL | GILPFVFTL | GILGFVFTL |
| GILGRVFTL | GILGDVFTL | GILGKVFTL | GILGPVFTL | GILGGVFTL |
| GILGFRFTL | GILGFDFTL | GILGFKFTL | GILGFPFTL | GILGFGFTL |
| GILGFVRTL | GILGFVDTL | GILGFVKTL | GILGFVPTL | GILGFVGTL |
| GILGFVFRL | GILGFVFDL | GILGFVFKL | GILGFVFPL | GILGFVFGL |
| GILGFVFTR | GILGFVFTD | GILGFVFTK | GILGFVFTP | GILGFVFTG |

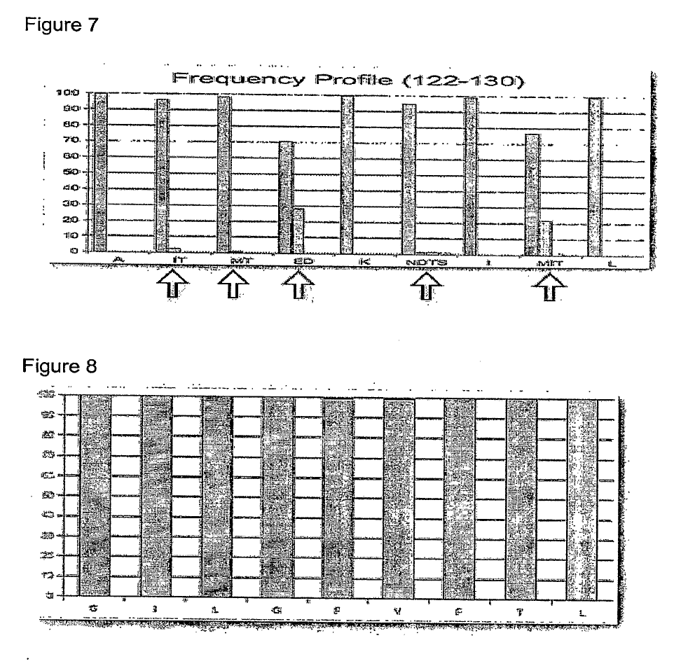

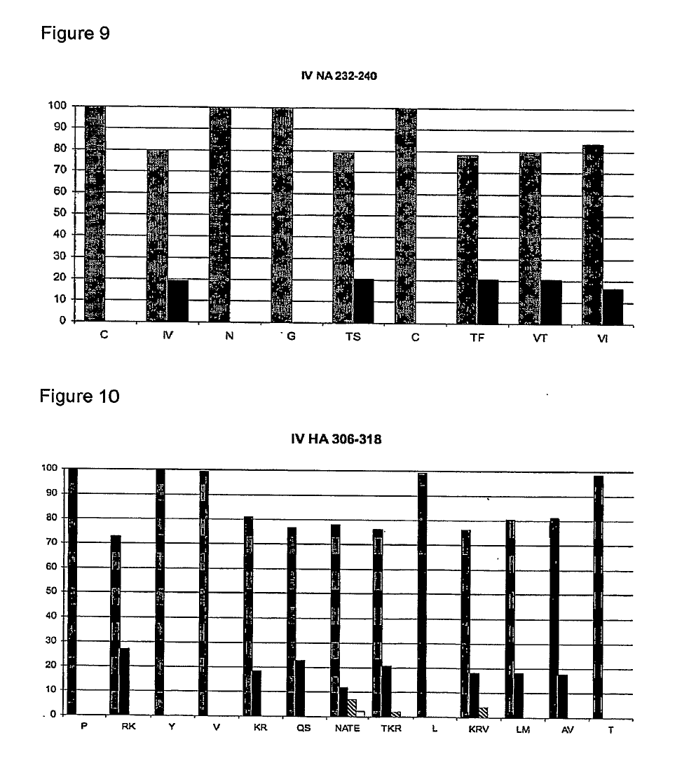

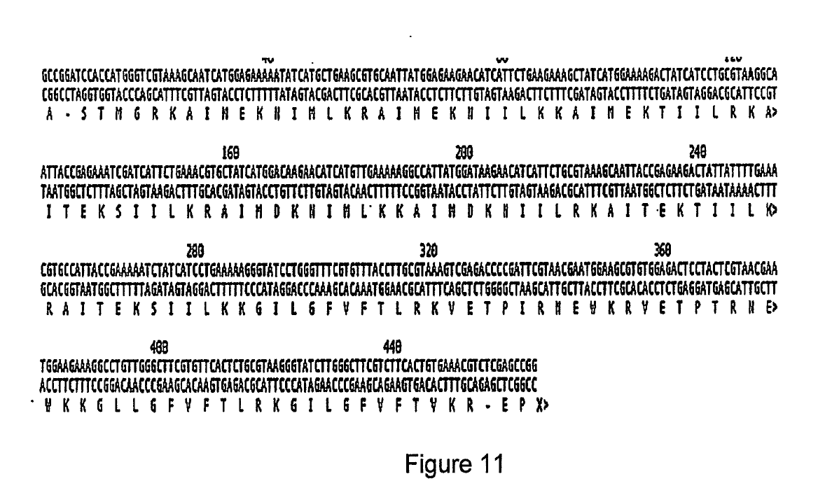

For the NS1 122-130 epitope, the observed amino acid variability between Influenza subtypes in that given HLA binding region was similarly determined. An epitope position is “variable” if there are at least two amino acid residues above a threshold value (ie. 5% occurrence). For example, all Influenza NS1 sequences from the LANL were compiled and aligned and generated the amino acid frequency distribution profile for the NS1 122-130 (FIG. 7).

| 122 | A | I | M | E | K | N | I | M | L | 130 |
|---|---|---|---|---|---|---|---|---|---|---|
| | | | T | D | | D | | I | | |
| | | | | | | T | | T | | |
| | | | | | | S | | | | |

At each of the variable peptide positions (124, 125, 127, and 129), we then enumerate the existing amino acid possibilities that then provide a matrix table to generate CBM variants. The possible combinatorial sequences are then:

AIMEKNIML
AIMEKNIIL
AIMEKDIML
AIMEKDIIL
AIMEKTIML
AIMEKTIIL
AIMEKSIML
AIMEKSIIL
AIMDKNIML
AIMDKNIIL
AIMDKDIML
AIMDKDIIL
AIMDKTIML
AIMDKTIIL
AIMDKSIML
AIMDKSIIL
AITEKNIML
AITEKNIIL
AITEKDIML
AITEKDIIL
AITEKTIML
AITEKTIIL
AITEKSIML
AITEKSIIL
AITDKNIML
AITDKNIIL
AITDKDIML
AITDKDIIL
AITDKTIML
AITDKTIIL
AITDKSIML
AITDKSIIL
AIMEKNIML
ATMEKNIIL
ATMEKDIML
ATMEKDIIL
ATMEKTIML
ATMEKTIIL
ATMEKSIML
ATMEKSIIL
ATMDKNIML
ATMDKNIIL
ATMDKDIML
ATMDKDIIL
ATMDKTIML
ATMDKTIIL
ATMDKSIML
ATMDKSIIL
ATTEKNIML
ATTEKNIIL
ATTEKDIML
ATTEKDIIL
ATTEKTIML
ATTEKTIIL
ATTEKSIML
ATTEKSIIL
ATTDKNIML
ATTDKNIIL
ATTDKDIML
ATTDKNIIL
ATTDKTIML
ATTDKTIIL
ATTDKSIML
ATTDKSIIL

### Positional Sequence Score Matrix (PSSM) Scores

After the CBM and LTM in silico mutagenesis of the starting wild-type NS1, M1 and M2 sequences, we then re-scored all the generated variants. The purpose is to retain for further testing only those variations that yield the higher than wild-type positional sequence score matrix (PSSM) scores. For this, we used the frequency scores of the entire (MHC-PEP) HLA-A*0201 listed peptides to generate a PSSM for each HLA binding position. For example, anchor sequences received a score of 10 while the other secondary positions (eg. 4 and 8) that demonstrate limited preferred amino acids were given a score of 5. In the remaining epitope positions, other recovered amino acids were then given a lower score (3, 2 or 1).

The PSSM then calculates a HLA score ranking their predicted binding affinity. In our PSSM examples, NS1 122-130 CBM variations; AIMEKNIIL (41), AIMDKNIML (42), AIMDKNIIL (41) scored higher than the wild type AIMEKNIML (37) sequence. The M1 58-66 LTM variant sequence GILGFVFTL (70) was predicted to have higher HLA affinity than the wild type GILGFVFTL (51).

### Example 3

### Combination Multi-Epitope Polypeptide Cloning and Microbial Synthesis

Construction of CME Expression Clones

The CME protein is constructed through a series of coding oligonucleotides (SEQ IDs 64-77) and ligation steps. Each “epitope” region is composed of a duplexed DNA with both a 5′ and 3′ overhanging single stranded ends that contain the Lys and or Arg codon (Lys/Arg) sequences. Particularized ordering of multiple target regions is achieved by incorporating unique Lys/Arg codons and flanking sequences during the SOE-PCR ligation assembly reaction. For example, the terminal 3′ end or “sticky end” of the first epitope region could incorporate the Lys/Arg codon sequences -aaa cgt-. The terminal 5′ end of following adjacent second epitope region would then have the requisite complementary -ttt gca- annealing sequence. In this case, the second epitope region 3′ Lys/Arg codon sequences would be -aag cgg- and is different from that of the first region's -aaa cgt-. The differences in Lys/Arg codon sequences then permits specific annealing with the 5′ complementary end of the third epitope region that has -ttc gcc- overhang. Here, the 5′ ttc gcc overhang of the third epitope region would not anneal to the 3′ aaa cgt overhang of the first epitope region.

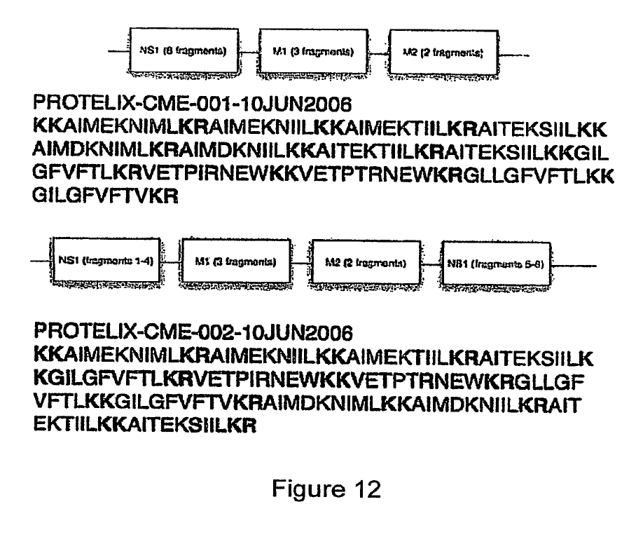

By creatively manipulating the available degenerate codon sequences for Lys/Arg in this manner then, the various other target epitopes can be precisely aligned in a tailored order (see FIG. 12). In this example there are two CME arrangements in ordering the NS1, M1 and M2 epitopes. The first CME illustrates putting together the eight NS1 variants at the N-terminus followed by the three M1 and two M2 variant epitopes. The second CME divides the eight NS1 epitope variants. The first four NS1 variants are together at the N-terminus of the CME peptide and the remaining four NS1 variants are at the C-terminus thereby sandwiching the three M1 and two M2 variants. The reason for varying the design IS to present different CME protein iterations into the test organism and determine which CME design may be more accessible to complete proteolytic cleavage to thereby release the individual epitopes.

Based on computer modeling programs, both CME constructs are predicted to fold primarily as an alpha helix with a small hydrophobic section attributable to the M1 epitope sequences. The accessibility of the intervening Lys/Arg cleavage sites can be tested by in vitro proteolysis with trypsin, chymotrypsin and other available enzymes. The digested fragments can then be analyzed by HPLC and mass spec to fingerprint the products and determine the actual cleavage sites for use in further re-design of the CME epitope order.

Microbial Expression and purification of Combination Multi-Epitope.

A major requirement for a successful synthetic protein-based vaccine is the ability to produce biologically active polypeptide that can be easily scaled up for mass production in economical organisms. The recombinant polypeptide should (i) possess compatible codons for high host expression, (ii) be non-toxic to production host and (iii) be biologically recognized as a proper immunogen by the test organism(s). Historically, Escherichia coli has been used for the expression of proteins in large amounts. However, due to the prokaryotic redox microenvironment of E. coli, it is sometimes difficult to produce correctly folded molecules. The eukaryotic yeast, Pichia pastoris, has been developed as an alternate expression host. Both of these microorganisms have been reported for use in trial vaccine production.

Construction of CME constructs are made based on both E. coli and Pichia optimized codons choosing not just the most frequently used codons, but also other frequently used codons to avoid depleting a given charged tRNA species. A carboxyl-terminal HisX6 tag to enable CME purification by Ni beads. The E. coli constructs will be transformed into BL21 host T-cells for Superbroth growth in fermentors then induced with 0.1 mM IPTG (isopropyl-D-thiogalactopyranoside). The P. pastoris constructs will be cloned into the pPIC3.5 vector (Invitrogen), a HIS4 selectable plasmid that contains an alcohol oxidase 1 promoter for growth in buffered glycerol medium. The yeast cells will be induced by growth in medium containing 1% methanol.

Collected E. coli or P. pastoris pellets can be disrupted by sonication whereupon, and centrifuged to pellet cell debris and fragments. The clarified supernatants are then loaded onto Ni-nitrilotriacetic acid (Ni-NTA) agarose (Qiagen) columns, washed extensively and CME protein eluted with variable concentrations of imidazole. The CME protein is further purified by ion-exchange and size exclusion chromatography to remove remaining contaminants and obtain a measure of its oligomeric state. The CME's are concentrated by membrane filtration, sterile filtered, quantified and stored frozen at −80 C or lyophilized for further use.

Mass Spectrophotometer Analysis:

The quality of our CME peptides can be analyzed using Matrix-Assisted Laser Desorption Ionization/Time-Of-Flight (Bruker-Daltronics) mass spectrometry. Mass spectra will be collected using our Autoflex II instrument. Observed spectra will be externally calibrated with known standards such as insulin, resulting in mass accuracy typically within ±0.1%.

### Example 4

### CME Vaccination of Control and HLA Transgenic Mice

Control C57BI/6 mice (6-12 weeks old) can be purchased from the Jackson Laboratory and maintained in a specific pathogen-free isolation environment.

The identification and in vivo testing of viral T cell immunodominant epitopes have direct application in human vaccine development. Transgenic mice expressing human HLA alleles have been developed to provide an animal model to study of HLA restricted CTL responses in human disease. By expressing human HLA-A*0201 (aka HLA-A2.1) alleles, HLA-A2.1 transgenic mice bind and present the same antigen-derived peptides as do allele-matched humans. These HLA-A2.1 transgenic are in the C57BI/6 genetic background have been shown to be susceptible to Influenza infection and will manifest morbidity and/or mortality outcomes.

These HLA-A2.1 transgenic mice have been used to identify HLA-A*0201 restricted epitopes recognized by human CTLs indicating that some antigen processing and recognition events are well-conserved between species. Thus, in vivo modeling of HLA epitopes that are immunogenic in HLA-A2.1 mice have very good correlation with likely human T cell immune responses.

CME Immunization.

The mice (6-8 weeks old) are divided into three groups with each group being composed of 4-5 mice. The negative control group will receive PBS adjuvant compositions and the positive control group will receive UV killed whole A/PR/8/34 virus. The CME vaccination groups will receive either low (10 μg) or high (100 μg) doses of the CME vaccine with adjuvant formulation. Intranasal immunization of vaccines would preferentially prime the resident immune cells in the lung and respiratory tract. This would stimulate the APCs in the primary mucosal areas where the virus is naturally introduced. Intranasal formulations have usually been composed of total virus protein vaccines. Alternatively, injecting the CME vaccine formulation by intraperitoneal, intramuscular, or sub-cutaneouse routes can deliver peptide antigens. Our CME formulation will be injected intramuscularly for the priming followed by two booster immunizations administered at 3 weeks intervals.

Influenza Antigen Specific Antibody Titer Analysis.

Blood collection and Influenza-specific antibody endpoint titer are collected before and after CME immunization. Specific HA and/or NA antibody determination is performed by serial dilution of the sera before application to 96-well enzyme-linked immunosorbent assay (ELISA) plates. The wells are plated with either detergent disrupted influenza virus or coated with the HA and/or NA vaccine candidate peptides and then blocked with (1% bovine serum albumin (BSA) in PBS. Blood samples are then added for approximately two hours before washing the plates for non-specific binding. After washing with 0.05% tween-20/PBS, the wells are treated with Horse Radish Peroxidase (HRP)-labeled anti-mouse IgG antibody. HRP substrate (3,3′-diaminobenzidine tetrahydrochloride dihydrate in 50 mM Tris.HCl, pH 7.5, containing 0.015% hydrogen peroxide) is then applied and OD values determined to calculate specific antibody dilution ranges.

### Example 5

### Preparation of Influenza Infection Stocks in Egg Allantoic Cavity or MDCK Infected Cells

Influenza viruses are grown in embryonated chicken eggs between the ages of 6 and 14 days old at 37° C. for 48 h. One hundred plaque forming units (pfu) of virus are injected into the allantoic cavity of each egg. Allantoic fluid from influenza virus-infected eggs are then serially diluted in PBS and assayed for hemagglutination (HA) of chicken red blood cells in 96-well plates. To determine the tissue culture infectious dose (TCID₅₀), plaque assays of the influenza virus stocks are performed on Madin-Darby canine kidney cells (MDCK) cells in the presence of 2 μg/ml trypsin at 37° C.

Alternatively, infectious stock virus can be prepared from dishes of 70% confluent MDCK cells are infected with Influenza A virus strains. The strains chosen are A/PR/8/34 (H1N1), A/SW/33 (H1N1), and A/HK/8/68 (H3N2).

### Example 6

### Preparation of Influenza Infection Stocks

Influenza Virus Challenge:

Preliminary studies will determine the viral dose response and pinpoint an optimal challenge dose prior to vaccination trials. Candidate mice (6-8 weeks old C57BI/6) will be infected intranasally with (10, 50, 100, 250, and 1×10³ TCID₅₀/ml) after which the mice morbidity and mortality curves will be established to calculate the minimum lethal dose MLD₅₀.

Three weeks after the last CME vaccination, the three groups of mice (CME vaccinated, negative and positive control) will be challenged. From the LD₅₀ determination, we will choose a viral dose that delivers a pathogenic response. At a low MLD₅₀ dose, all the animals should become ill and yield discernable morbidity measurements but not become so sickened that 100% of even the positive control group dies.

Influenza Virus Strain A/Puerto Rico/8/34:

We will initially use Influenza A/ Puerto Rico/8/34 (H1N1) as the challenge strain because it is a mouse adapted strain. More importantly, the initial NS1₁₂₂₋₁₃₀ epitope sequence was recovered from A/PR/8/34 challenge to HLA-A2.1 transgenic mice. Thus, it would be expected that the same known HLA-A*0201 presented NS1₁₂₂₋₁₃₀ epitope from A/PR/8/34 virus would confer some CTL cellular immunoprotection against the same A/PR/8/34 homotypic challenge strain. Moreover, the HLA-A2.1 transgenic mice are also very likely to respond to the same HLA-A*0201 restricted M1₅₈₋₆₆ epitope.

Heterotypic Challenge with Other Influenza Virus Strains:

An important consideration in evaluating vaccine compositions is protection from further infections by differing immunogenic strains. The CME we will be assessed for its capacity to cross-protect against other H1N1 strains such as A/WS/33 and heterotypic influenza viruses of the serologically distinct A/AA6/60 (H2N2) and A2/Hong Kong/8/68 (H3N2) subtypes.

### Example 7

### Determination of CME Vaccine Protective Response after Virus Challenge

Survival and Weight Loss Pattern after Virus Challenge

For total respiratory tract infection, the test mice will be anesthetized with isoflurane so that a small volume of liquid can be administered into each nostril flare. Most studies rely on intranasal inoculation of influenza during the challenge phase, as this route causes mice to develop an illness closely resembling the human disease. There is a progressive spread of the virus from the upper to the lower respiratory tract. Mice will be followed on a daily basis for three weeks post-viral challenge. The two parameters often used to assess the protective effects of vaccines are survival (mortality) and weight loss (morbidity). As a measure of disease course, both mortality and morbidity can be determined concurrently using the same group of mice. Morbidity is assessed by body weight, calculated as a percentage of the original body weight at the time of infection, and monitored on a daily basis for 3 weeks. If the mice display severe signs of sickness, meaning they lose more than 25% of their body weight, they will be euthanized to prevent further suffering as required by NIH guidelines. Mice generally cannot recover from a weight loss this substantial, and eventually succumb to complications arising from the infection.

Measuring Virus Spread and Clearance from Organs Following Infection:

To assess the vaccine's protective efficacy, we will measure viral titers from various tissues to determine the amount of Influenza spread within the host mice. A set of mice will be killed at either day 3 or day 6 post infection. Organ tissue from the spleen, liver, kidney will be collected and homogenized. The supernatant and dilutions thereof are in sterile PBS (100 mg lung tissue per 1 ml PBS) and quantitated on MDCK TCID₅₀ titer assays.

### Example 8

### Assessing Vaccine Efficiency Through Subsequent Immunological Analyses

T-cells involved in conferring protective immunity can include both CD4+ and CD8+ T-cells. Therefore we must determine whether a primarily induced expansion of CD8+ T lymphocytes and/or CD4+ T-cell responses. We will evaluate the immunogenecity of our optimized epitopes in the HLA-A*0201 transgenic mice. Other studies have demonstrated that generally most, but not all, high affinity HLA binders will yield epitope specific responses. In fact, since these NS1, M1 and M2 epitopes are known in vivo HLA-A*0201 presented peptides they are more than likely to have elicited an immunogenic reaction. These antigen specific effects can be monitored by a variety of techniques; CD8+ CTL response, epitope specific T cell proliferation assays, and ELISPOT.

Influenza Epitope CTL Responses from Mice Splenocytes:

CTL is the primary mechanism by which mice recover from viral infection. Activated CD8+ CTLs generally kill any cells that display the specific peptide:MHC complex they recognize. Hence, following infection, specific CTLs are generated to recognize and kill Influenza infected cells. To demonstrate specificity of anti-Influenza CTL in vitro, splenocytes from CME vaccinated mice are collected and induced to become CTL effector cells. The negative control will be splenocytes generated from the mock vaccine.

Total spleen cells from Influenza infected mice are collected and re-stimulated in vitro for two 7-day periods. Splenocytes are divided into separate wells so that they can be individually incubated with the various epitopes in media [supplemented with 0.5 U/ml of mouse IL-2 (Life Technologies). Autologous peptide-pulsed spleen cells are the source of APCs and T cells for the first time period. For the second period, viable cells are harvested and restimulated with peptide-pulsed irradiated (2000 rad) strain-matched spleen cells (C57BI/6) that serve as APCs.

These target cells are then labeled with Na₂⁵¹CrO₄ and pulsed with various concentrations of the test HLA-A*0201 NS1, M1 and M2 epitope peptides. After 4 hours of incubation of effectors with targets at various E:T ratios. Typical Effector: Target ratios are 100:1, 50:1, 25:1, and 12.5:1. Supernatants are harvested and counted on a gamma counter. Specific APC lysis is then calculated as:

((experimental−spontaneous release)/(maximal−spontaneous release))×100%.

It is important to demonstrate HLA epitope specificity using primed anti-influenza CTL activity. CTLs from the CME vaccinated mice should lyse only those APC cells sensitized by peptide loading with the individual NS1 and M1 epitopes. We expect that once the CME peptide construct is introduced into the HLA-A*0201 transgenic mice, the contiguous peptide will be proteolytically cleaved (intracellularly and/or extracellularly) releasing the component epitopes. The separate NS1 peptides (i.e., AIMEKNIIL, 2AIMDKNIML, 3AIMDKNIIL and wild type NS1 4AIMEKNIML) are added into separate wells to be loaded, and presented by APC target cells. The subsequent CTL assays are informative to demonstrate which of the CME epitopes were in vivo immunodominant. Some and maybe all of the NS1 and M2 HLA-A*0201 restricted epitopes will then be presented on APC surface to stimulate in vivo CD8+ effector and memory cells. It is expected that the M2₇₋₁₅ epitope specific for HLB-44 will not presented in these HLA-A*0201 mice and therefore not elicit CD8+ CTLs and memory. Therefore, only NS1 and M1 epitopes that generate in vivo CD8+ CTLs and memory cells are then expanded by in vitro stimulation.

Interferon γ ELISPOT Assay

One method to measure the responses of T-cell populations is a variant of the antigen-capture ELISA method, called the ELISPOT assay. In this assay, cytokine secreted by individual activated T cells is immobilized as discrete spots on a plastic plate via anti-cytokine antibodies, which are counted to give the number of activated T cells. Enzyme-linked immunorsorbent spot (ELISPOT) can then measure the production of various cytokines from antigen specific stimulated T-cells. The assay essentially captures secreted cytokine on a filter bottom. Ninety-six-well nitrocellulose plates (Milititer HA, Millipore) are coated with 7 μg/ml of a monoclonal antibody against mouse IFN-gamma (PharMingen) in 75 μl PBS and incubated at room temperature overnight. Wells are washed six times with culture medium and incubated with DMEM supplemented with 10% FCS for 1 h at 37° C. Splenocytes from immunized and/or challenged mice are then added to antibody-coated wells in serial dilutions. The recovered splenocytes are activated ex vivo with autologous target cells pulsed with the various individual epitope peptides starting with a dilution of 1×10⁻⁶ M of the synthetic CME candidate peptide (see Seq Ds above).

Briefly, after overnight incubation at 4° C., the wells are washed and blocked with culture medium containing 10% fetal bovine serum. Splenocytes (1×10⁶/well along with 15 IU/ml IL-2), are added to the well and incubated at 37° C. for 24 h either with or without the test epitopes. After culture, the plate was washed and then followed by incubation with 5 μg/ml biotinylated IFN-antibody (clone XMG1.2, PharMingen). After washing, 1.25 μg/ml avidin-alkaline phosphatase (Sigma) is added and incubated for 2 hours and washed again. The spots are then developed by adding 5-bromo-4-chloro-3-indolyl phosphate/nitroblue tetrazolium solution (Boehringer Mannheim) and counted using a the aid of a dissecting microscope. The frequency of epitope-specific cells was determined from the difference between the number of spots seen with and without epitope peptide during re-stimulation.

The main advantage with ELISPOT is that it is very high throughput and it simply measures cytokine secretion. The disadvantage is that one cannot determine whether cytokine positive cell is a CD4+, CD8+ or other type of cell.

Intracellular Cytokine Response Staining:

Intracellular cytokine staining (ICS) is a widely used technique in flow cytometry: it is able to detect production and accumulation of cytokines in the endoplasmic reticulum following stimulation. Multi-color FACS analysis allows the detailing which population frequency, both CD8+ and CD4+ cells, and the cytokines, IFN-γ, TNF-β, IL-2, IL-4, IL-5, IL-6, IL-10, and IL-13, they are secreting in response to viral infection. After peptide vaccination for example, increased IFN-γ production can be determined to be the result of CD8+ stimulation or CD4+ which would aid in targeting epitope design.

As above, the cells are stimulated with the epitope peptide and extra-cellular release of the cytokine is blocked by the addition of an export inhibitor increasing intracellular cytokine concentration. The cells are then permeabilized to allow staining by cytokine specific antibodies. Briefly, splenocytes from mock control or vaccinated groups of mice are separately incubated with the above individual test NS1, M1 and M2 epitopes. Golgistop (PharMingen) is added 6 hours before cell harvesting whereupon they are washed in FACScan buffer and stained with phycoerythrin-conjugated monoclonal rat antimouse CD8 or CD4 antibody (PharMingen). Cells are then subjected to intracellular cytokine staining using the Cytofix/Cytoperm kit (PharMingen) FITC-conjugated anti-IFN-γ and assayed using flow cytometry.

MHC-Peptide Tetramer Analysis:

Tetramer staining is often combined with intracellular cytokine cytometry. Tetramers are soluble complexes of four MHC molecules associated with the peptide epitope. The advantage of doing the two together is the ability to see which cells are actually functional in terms of making a cytokine to that particular epitope. In most other applications, cell populations with heterogenous HLA expression, the drawback is that it only works for one specific epitope at a time and a different HLA specific tetramer is needed for every single epitope. However, for our purposes, since we are using HLA-A*0201 transgenic mice, the HLA specificity has been predetermined and we only need to prepare HLA-A*0201 tetramers.

Proliferation Assay (3HTdR Incorporation into DNA):

Target cells are irradiated and incubated together with peptide-specific T-cells at various effector:target ratios. At certain time points, ³H thymidine is added to the culture and after overnight growth and DNA incorporation, cells are lysed and the radioactivity is measured as an indication of the amount of proliferation of the T-cell population.

Serologic Tests

If the mice above are able to produce anti-influenza specific antibodies, the protective nature of these antibodies can be assayed using MDCK neutralization assays. Neutralization assays were done by mixing 100 ID50 of virus (strain of choice) and test antisera for 1 h at 23° C.; this is followed by titration of the mixtures for residual virus infectivity on MDCK cell monolayers in 96-well plates. After 3 days of incubation at 37° C. in 5% CO2, neutralization titers were assessed for the presence of a cytopathic effect in the cultures and for HA activity in the supernatant. Neutralization titers are then expressed as the reciprocal of the antibody dilution that completely inhibited virus infectivity in 50% of triplicate cultures.

| | |
|---|---|
| SEQ ID NO: 1 | MSLLTEVETPTRNGWECKCSDSSDPLVIAASIIGILH |
| | LILWILDRLFFKCIYRRLKYGLKRGPSTEGVPESMRE |
| | EYRQEQQSAVDVDDGHFVSIELE |
| SEQ ID NO: 2 | MSFLTEVETPIRNEWGCRCNGSSDPLTIAANIIGILH |
| | LTLWMLDRLFFKCIYRRFKYGLKGGPSTEGVPKSMRE |
| | EYRKEQQSAVDTDDGHFVSIELE |
| SEQ ID NO: 3 | MSLLTEVETPTKNGWECRCSDSSDPLVIAASIIGILH |
| | LILWILDRLFFKCIYRRLKYGLKRGPSTEGVPESMRE |
| | EYRQEQQSAVDVDDGHFVNIELE |
| SEQ ID NO: 4 | MSLLTEVETPTRNEWECRCSDSSDPLVVAASIIGILH |
| | LILWILDRLFFKCIYRRLKYGLKRGPSTAGVPESMRE |
| | EYRQEQQSAVDVDDGHFVNIELE |
| SEQ ID NO: 5 | MSLLTEVETPTRNEWECRCSDSSDPLVVAASIIGILH |
| | LILWILDRLFFKCIYRRLKYGLKRGPSTAGVPESMRE |
| | EYRQEQQSAVDVDDGHFVNIELE |
| SEQ ID NO: 6 | MSLLTEVETPTRNGWECKCSDSSDPLVIAASIIGILH |
| | LILWILDRLFFKCIYRRLKYGLKRGPSTEGVPESMRE |
| | EYRQEQQSAVDVDDGHFVNIELE |
| SEQ ID NO: 7 | MSLLTEVETPTRNGWECKCSDSSDPLVIAASIIGILH |
| | LILWILDRLFFKCIYRRLKYGLKRGPSTEGVPESMRE |
| | EYRQEQQSAVDVDDSIIFVNIELE |
| SEQ ID NO: 8 | MSLLTEVETPTRNGWECKCSDSSDPLVVAASIIGILH |
| | LILWILDRLFFKCIYRRKYGLKRGPSTEGVPESMREE |
| | YRQEQQSAVDVDDGHFVNIELE |
| SEQ ID NO: 9 | MSLLTEVETPTRNGWGCRCSGSSDPLVVAASIXGILN |
| | LILWILDRLFFKCIYRRFKYGLKRGPSTEGVPESMRE |
| | EYRQEQQSAVDVDDGHFVNIELE |
| SEQ ID NO: 10 | MSLLTEVETPTRNGWECRCSDSSDPLVIAASIIGILH |
| | LILWILDRLFFKCIYRRLKYGLKRGPSTGGVPESMRE |
| | EYRQEQQSAVDVDDGHFVNIELE |
| SEQ ID NO: 11 | MSLLTEVETPTRNGWECRCSDSSDPLVIAASIIGILH |
| | LILWILDRLFFKCIYRRLKYGLKRGPSTEGVPESMRE |
| | EYRQEQQSAVDVDDGHFVNIELE |
| SEQ ID NO: 12 | MSLLTEVETPTRNGWECRCSDSSDPLVIAASIIGILH |
| | LILWILDRLFFKCIYRRLKYGLKRGPSTEGVPESMRE |
| | EYRQEQQNAVDVDDGHFVNIELE |
| SEQ ID NO: 13 | MSLLTEVETHTRSGWECRCSDSSDPLVIAASIIGILH |
| | LILWILDRLFFKCIYRRLKYGLKSGPSTEGVPESMRE |
| | EYQQEKQSAVDVDDGHFVNIELE |
| SEQ ID NO: 14 | MSLLTEVETPTRNEWECKCSDSSDPLVIAASIIGILH |
| | LTLWILDRLFFKCIYRRLKYGLKRGPSTEGVPESMRE |
| | EYRQEQQNAVDVDDGHFVNIELE |
| SEQ ID NO: 15 | MSLLTEVETPTRNGWECKCSDSSDPLVIAASIIGILH |
| | LILWILDRLFFKCIYRRLKYGLKRGPSTEGVPESMRE |
| | EYRQEQQSAVDVDDGHFVNIELE |
| SEQ ID NO: 16 | DPL Xaa1 Xaa2 AA Xaa3 I where Xaa1 is |
| | selected from the group consisting of |
| | V and T; Xaa2 is selected from the |
| | group consisting of I and V; and Xaa3 |
| | is selected from the group consisting |
| | of S and N. |
| SEQ ID NO: 17 | Xaa1 Xaa2 Xaa3 W Xaa4 C Xaa5 C XaaG |
| | Xaa7 SSDPL where Xaa1 is selected |
| | from the group consisting of R and K; |
| | Xaa2 is selected from the group |
| | consisting of N and S; and Xaa3 is |
| | selected from the group consisting of |
| | G and E; Xaa4 is selected from the |
| | group consisting of E and C; and Xaa5 |
| | is selected from the group consisting |
| | of R and K; Xaa6 is selected from the |
| | group consisting of S and N; and Xaa7 |
| | is selected from the group consisting |
| | of D and C. |
| SEQ ID NO: 18 | DSNTVSSFQVDCFLWHVRKRFADQELGDAPFLaRLRR |
| | DQKSLRGRGSTLGLDIrTATheGKhIVERILEEESDE |
| | ALKMTIASVPApRYLTDMTLEEMSRDWlMLiPKQKVt |
| | GSLCIRMDQAIvDKNITLKANFSVIFnRLEALILLRA |
| | FTdEGAIVGEISPLPSLPGHTDEDVKNAIGiLIGGfE |
| | WNDNTVRVSETLQRFAWRSSdEDGRPPLsPKeKReMe |
| | RTIEpEV |
| SEQ ID NO: 19 | MDSNTVSSFQVDCFLWHVRKRFADQELGDAPFLDRLR |
| | RDQKSLRGRGnTLGLDIETATcAGKQIVERILEEESD |
| | EALKM-----PApRYLTDMTLEEMSRDWFMLMPKQKV |
| | AGSLCIkMDQAIMDKNIILKANFSVIFDRLETLILLR |
| | AFTEEGAIVGEISPLPSLPGIITDEDVKMAIGVLIGG |
| | LEWNDNTVRVSETLQRFAWRSSdEDGRPPLPPnQKRK |
| | MARTIESEV |
| SEQ ID NO: 20 | FQVDCFLWHVRXRFADQELGDAPFLDRLRRDQKSLRG |
| | RGSTLGLDIETATRAGKQIVERILEEESDEALKMTIA |
| | SVPASRYLTDMTLEEMSRDWFMLMPKQKVAGSLCIRM |
| | DQAIMDKNIILKANFSVIFDRLETLILLRAFTEEGAI |
| | VGEISPLPSLPGHTDEDVKISIAIGVLIGGLEWNDNT |
| | VRVSETLQRFAWRSSNEDGRPPLPPKQKRKMARTIES |
| | EV |
| SEQ ID NO: 21 | MDSNTVSSFQVDCFLWHVRKRFADQELGDAPFLDRLR |
| | RDQKSLRGRGSTLGLDIdTATRAGKQIVERILEEESD |
| | EALKMTItSVPASRYLTDMTLEEMSRDWFMLMPKQKV |
| | AsSLCIRMDQAIMDKNIILKANFSVIFDRLEaLILLR |
| | AFTEEGAIVGEISPLPSLPGHTDEDVKNAIeVLIGGL |
| | EWNDNTVRVSETLQRFAWRScNEnGRPPLPPKQKRKM |
| | ARTIESEV |
| SEQ ID NO: 22 | MDSNTVSSFQVDCFLWHVRKRFADQELGDAPFLDRLR |
| | RDQKSLRGRGSTLGLDIETAThAGKQIVERILEEESD |
| | EALKMTIASVPASRYLTDMTLEEMSRDWFMLMPKQKV |
| | AGSLCIRMDQAIMDKNIILKANFSVIFDRLETLILLR |
| | AFTEEGAIVGEISPLPSLPGHTDEDVKNAIGVLIGGL |
| | EWNDNTVRVSETLQRFAWRSSNEDGRPPLPPKQKRKM |
| | ARTIESEV |
| SEQ ID NO: 23 | MDSNTVSSFQVDCFLWHVRKRFADQELGDAPFLDRLR |
| | RDQKSLRGRGSTLGLDIETATRAGKQIVERILEEESD |
| | EALKMTItSVPASRYLTDMTLEEMSRDWFMLMPKQKV |
| | AGSLCIRMDQAIMDKDIIkLKANFSVIFDRLETLILL |
| | RAFTEEGAIVGEISPLPSLPGHTDEDVKNAIGVLIGG |
| | LEWNDNTVRVSETLQRFAWRSSNEDGRPPLPPKQKRK |
| | MARTIESEV |
| SEQ ID NO: 24 | MDSNTVSSFQVDCFLWHVRKRFSADQELGDAPFLDRL |
| | RRDQKSLRGRGSTLGLDIETATRAGKQIVERILEEES |
| | DEALKMTIASVPtSRYLTDMTLEEMSRDWFMLMPKQK |
| | VAGSLCIRMDQAIMDKNIILKANFSVIFDRLETLILL |
| | RAFTEEGAIVGEISPLPSLPGHTDEDVKNAIGVLIGG |
| | LEWNDNTVRVSETLQRFAWRSSNEDORPPLPPKQKRK |
| | MARTIESEV |
| SEQ ID NO: 25 | MDSNTVSSFQVDCFLWHVRKRFADQELGDAPFLDRLR |
| | RDQKSLRGRGSTLGLDIETATRAGKQIVERILEEESD |
| | EALKMTIASVPASRYLTDMTLEEMSRDWFMLMPKQKV |
| | AGSLCIRMDQAIMDKNIILKANFSVIFDRLETLILLR |
| | AFTEEGAIVGEISPLPSLPGHTDEDVKNAIGVLIGGf |
| | EWNDNTVRVSETLQRFAWRSSNEnGRPPLPPKQKRKM |
| | ARTIESEV |
| SEQ ID NO: 26 | MDSNTVSgFQVDCFLWHVRKRFADQELGDAPFLDRLR |
| | RDQKSLRGRGSTLGLDIETATRAGKQIVERILEEESD |
| | EALqMTIASVPASRYLTDMTLEEMSRDWFMLMPKQKV |
| | AGSLCIRMDQAIMDKNIILKANFSVIFDRLETLILLR |
| | AFTEEGAIVGEISPLPSLPGHTDEDVKNAIGVLIGGL |
| | EWNDNTVRVSETLQRFAWRSSNEDGRPPLPPKQKRKM |
| | ARTIESEV |
| SEQ ID NO: 27 | MDSNTitSFQVDCyLWHiRKllsmrdmcDAPFdDRLR |
| | RDQKaLkGRGSTLGLDlrvATieGKkIVEdILksEpD |
| | EiLKiaIASiPApRYiTDMsmEEiSReWyMLMPrQKi |
| | tGgLvvkMDQAIMDKRIILKANFSVlFnqLETLvSLR |
| | AFTddGsIVaEISPfPSmPGHsaEDVKNAIGiLIGGL |
| | EWNDNsVRaSEniQRFAWgvcdEnrgPsLPPnQKcyM |
| | AgrvESk |
| SEQ ID NO: 28 | MDSNTVSSFQVDCFLWHVRKRFADQELGDAPFLDRLR |
| | RDQKSLRGRGSTLGLDIETATRAGKQIVERILEEESD |
| | EALKMTIASVPASRYLTDMTLEEMSRDWFMLMPKQKV |
| | AGSLCIRMDQAIMDKNItLKANFSVIFDRLETLTLLR |
| | AFTdEGAIVGEISPLPSLPGHTDEDVKNAIGVLIGGL |
| | EWNDNTVRVSETLQRFAWRSSNEDGRPPLPPKQKRKM |
| | ARTvESEV |
| SEQ ID NO: 29 | MDSNTVSSFQVDCFLWHVRKRFADQGLGDAPFLDRDR |
| | RDQKSLRGRGSTLGLDIETATRAGKQIVERILEEESD |
| | EALKMTIASVPASRYLTDMTLEEMSRDWFMLMPKQKV |
| | AGSLCIRMDQAIMDKNIILKANFSVIFDRLETLILLR |
| | AFTEEGAIVGEISPLPSLPGHTDEDVKNAIGVLIGGL |
| | EWNDNTVRVSETLQRFAWRgSNEDGRPPLPPKQKRKM |
| | ARTIESEV |
| SEQ ID NO: 30 | MDSNTVSSFQVDCFLWHVRKRFADQdmGDAPFLDRiR |
| | RDQKSLkGRsiTiGmDIEaATRAGKQIiERILdEESD |
| | kALKMnIASVPApRYvTDMTPEEMSRDWFMLMPKQKf |
| | AGpLCIRMDQAIlDKNIILKANPSVvFDRLETLILLR |
| | AFTsEGAIVGEISQLPSLPGHTnEDVKNAIGiLIGGL |
| | EWNDNTVRVSETLQRFAWgSSNEnGRPPfaPKQeRKM |
| | AgTvESEV |
| SEQ ID NO: 31 | MDpNTVSSFQVDCFLWHVRKRvADQELGDAPFLDRLR |
| | RDQKSLRGRGSTLGLDIkTATRAGKQIVERILkEESD |
| | EALKMTmASVPASRYLTDMTLEEMSRDWSMLiPKQKV |
| | AGpLCIRMDQAIMDKNIILKANFSVIFDRLETLILLR |
| | AFTEEGAIVGEISPLPSLPGHTaEDVKNAvGVLIGGL |
| | EWNDNTVRVSETLQRFAWRSSNEnGRPPLtPKQKReM |
| | AgTIrSEV |
| SEQ ID NO: 32 | MDShTVSSFQVDCFLWHVRKqvADkdLGDAPFLDRLR |
| | RDQKSLkGRGSTLGLnIETATcvGKQIVERILkEESD |
| | EAFKMTmASALASRYLTDMTvEEMSRDWFMLMPKQKV |
| | AGpLCvRMDQAIMDKNIILKANFSVIFDRLENLTLLR |
| | AFTEEGAIVGEISPLPSFPGHTnEDVKNAIGVLIGGL |
| | EWNDNTVRVSETLQRFAWRSSNEtGgPPftttQKRKM |
| | AgTIrSEV |
| SEQ ID NO: 33 | MDSNTVSSFQVDCFLWHVRKRFADQELGDAPFLDRLR |
| | RDQKSLRGRGSTLGLDIETATRAGKQIVERILEEESD |
| | EALKMTIASVPASRYLTDMTLEEMSRDWFMLMPKQKV |
| | AGSLCIRMDQAIMDKNIILKANFSVIFgRLETLILLR |
| | AFTEEGAIVGEISPLPSLPGHTDEDVKNAIGVLIGGL |
| | EWNDNTVRVSETLQRFAWRSSNEDGRPPLPPKQKRKM |
| | ARTIESEV |
| SEQ ID NO: 34 | IF Xaa1 RLE Xaa2 L Xaa3 where Xaa1 is |
| | selected from the group consisting of |
| | D, G and N; Xaa2 is selected from the |
| | group consisting of T, A, and N; and |
| | Xaa3 is selected from the group |
| | consisting of I and T. |
| SEQ ID NO: 35 | D Xaa1 Xaa2 Xaa3 K Xaa4 Xaa5 Xaa6 |
| | Saa7 S Xaa8 Xaa9 A Xaa10 R where Xaa1 |
| | is selected from the group consisting |
| | of E and K; Xaa2 is selected from the |
| | group consisting of A and I; Xaa3 is |
| | selected from the group consisting of |
| | L and F; Xaa4 is selected from the |
| | group consisting of M and I; Xaa5 is |
| | selected from the group consisting of |
| | T, N and A; Xaa6 is selected from the |
| | group consisting of I and M; Xaa7 is |
| | selected from the group consisting of |
| | A and T; Xaa8 is selected from the |
| | group consisting of V, I and A; Xaa9 |
| | is selected from the group consisting |
| | of P and L; Xaa10 is selected from |
| | the group consisting of S and P. |
| SEQ ID NO: 36 | RPLVIAASI |
| SEQ ID NO: 37 | DRLVIAASI |
| SEQ ID NO: 38 | DPRVIAASI |
| SEQ ID NO: 39 | DPLRIAASI |
| SEQ ID NO: 40 | DPLVRAASI |
| SEQ ID NO: 41 | DPLVIRASI |
| SEQ ID NO: 42 | DPLVIARSI |
| SEQ ID NO: 43 | DPLVIAARI |
| SEQ ID NO: 44 | DPLVIAASR |
| SEQ ID NO: 45 | CGCCCGCTGGTGATCGCCGCCAGCATC |
| SEQ ID NO: 46 | GACCGCCTGGTGATCGCCGCCAGCATC |
| SEQ ID NO: 47 | GACCCGCGCGTGATCGCCGCCAGCATC |
| SEQ ID NO: 48 | GACCCGCTGCGCATCGCCGCCAGCATC |
| SEQ ID NO: 49 | GACCCGCTGGTGCGCGCCGCCAGCATC |
| SEQ ID NO: 50 | GACCCGCTGGTGATCCGCGCCAGCATC |
| SEQ ID NO: 51 | GACCCGCTGGTGATCGCCCGCAGCATC |
| SEQ ID NO: 52 | GACCCGCTGGTGATCGCCGCCCGCATC |
| SEQ ID NO: 53 | GACCCGCTGGTGATCGCCGCCAGCCGC |
| SEQ ID NO: 54 | DPLVIAASR |
| SEQ ID NO: 55 | DPLVSAASI |
| SEQ ID NO: 56 | DPLVIAASY |
| SEQ ID NO: 57 | DPLVIAASY |
| SEQ ID NO: 58 | DPLTIAASI |
| SEQ ID NO: 59 | DPLVVAASI |
| SEQ ID NO: 60 | DPLTVAASI |
| SEQ ID NO: 61 | DPLTIAANI |
| SEQ ID NO: 62 | DPLVVAANI |
| SEQ ID NO: 63 | DPLTVAANI |
| SEQ ID NO: 64 | GCCGGATCCACCATGGGTCGTAAGCAATCATGGAGAA |
| | AAATATCATGCTGAAGCGTGCATTATGGAGAAGAACA |
| | TCATTCTGAAGAAA |
| SEQ ID NO: 65 | GCTATCATGGAAAAGACTATCATCCTGCGTAAGGCCA |
| | ATTACCGAGAAATCGATCATTCTGAAACGT |
| SEQ ID NO: 66 | GCTATCATGGACAAGAACATCATGTTGAAAAAGGCCA |
| | TTATGGATAAGAACATCATTCTGCGTAAA |
| SEQ ID NO: 67 | GCAATTACCGAGAAGACTATTATTTTGAAACGTGCCA |
| | TTACCGAAAAATCTATCATCCTGAAAAAG |
| SEQ ID NO: 68 | GGTATCCTGGGTTTCGTGTTTACCTTGCGTAAAGTCG |
| | AGACCCCGATTCGTAACGAATGGAAGCGT |
| SEQ ID NO: 69 | GTGGAGACTCCTACTCGTAACGAATGGAAGAAACCTG |
| | TTGGGCTTCGTGTTCACTCTGCGTAAG |
| SEQ ID NO: 70 | GGTATCTTGGGCTTCGTCTTCACTGTGAAACGTCTCG |
| | AGCCGG |
| SEQ ID NO: 71 | CAGAATGATGTTCTTCTCCATAATTGCACGCTTCAGC |
| | ATGATATTTTTCTCCATGATTGCTTTACGACCCATGG |
| | TGGATCCGGC |
| SEQ ID NO: 72 | CAGAATGATCGATTTCTCGTAATTGCCTTACGCAGGA |
| | TGATAGTCTTTTCCATGATAGCTTTCTT |
| SEQ ID NO: 73 | CAGAATGATGTTCTTATCCATAATGGCCTTTTTCAAC |
| | ATTGATGTTCTTGTCCATGATAGCACGTTT |
| SEQ ID NO: 74 | CAGGATGATAGATTTTTCGGTAATGGCACGTTTCAAA |
| | ATAATAGTCTTCTCGGTAATTGCTTTACG |
| SEQ ID NO: 75 | CCATTCGTTACGAATCGGGGTCTCGACTTTACGCAAG |
| | GTAAACACGAAACCCAGGATACCCTTTTT |
| SEQ ID NO: 76 | CAGAGTGAACACGAAGCCCAACAGGCCTTTCTTCCAT |
| | TCGTTACGAGTAGGAGTCTCCACACGCTT |
| SEQ ID NO: 77 | CCGGCTCGAGACGTTTCACAGTGAAGACGAAGCCCAA |
| | GATACCCTTACG |
| SEQ ID NO: 78 | AIMEKNIML |
| SEQ ID NO: 79 | AIMEKNIIL |
| SEQ ID NO: 80 | AIMEKTIIL |
| SEQ ID NO: 81 | AITEKSIIL |
| SEQ ID NO: 82 | AIMDKNIML |
| SEQ ID NO: 83 | AIMDKNIIL |
| SEQ ID NO: 84 | AITEKTIIL |
| SEQ ID NO: 85 | AIMEKSIIL |
| SEQ ID NO: 86 | GILGFVFTL |
| SEQ ID NO: 87 | GLLGFVFTL |
| SEQ ID NO: 88 | GLLGFVFTV |
| SEQ ID NO: 89 | VETPIRNEW |
| SEQ ID NO: 90 | VETPTRNEW |
| SEQ ID NO: 91 | IF Xaa1 RLE Xaa2 LI where Xaa1 is |
| | selected from the group consisting of |
| | D, G and N; Xaa2 is selected from the |
| | group consisting of T, A, and N. |
| SEQ ID NO: 92 | D Xaa1 Xaa2 Xaa3 Xaa4 K Xaa4 A S Xaa4 |
| | Xaa5 Xaa6 Xaa7 S XaaB Xaa9 A Xaa10 R |
| | where Xaa1 is selected from the group |
| | consisting of E and K; Xaa2 is |
| | selected from the group consisting of |
| | A and I; and Xaa3 is selected from |
| | the group consisting of L and F; Xaa4 |
| | is selected from the group consisting |
| | of M and I; and Xaa5 is selected from |
| | the group consisting of T, N and I; |
| | Xaa6 is selected from the group |
| | consisting of I and M; and Xaa7 is |
| | selected from the group consisting of |
| | A and T; Xaa8 is selected from the |
| | group consisting of V, I and A; Xaa9 |
| | is selected from the group consisting |
| | of P and L; Xaa10 is selected from |
| | the group consisting of S and P; |

It is claimed:

1. A method of producing a vaccine or therapeutic composition against infection by a virus having a plurality of subtypes whose protein sequences for at least one viral protein or polyprotein are known, said method comprising
(a) for the at least one viral protein or polyprotein in at least one viral subtype, identifying a viral protein-sequence region of known or predicted binding to one or more selected MHC Class I or Class II proteins,
(b) comparing aligned protein sequences of the viral-protein sequence region identified in step (a) within the plurality of viral subtypes, to identify a candidate peptide whose sequence variation among the viral subtypes includes one of: (i) a string of at least eight contiguous amino acids at which substantially no sequence variation occurs, and (ii) a string of at least eight contiguous amino-acid positions having at least three amino acids at which amino acid substitution variations occur above a given threshold frequency,
(e) generating a set of candidate antigens having one of (ci) each of a plurality of defined-residue amino acid substitutions at each of a plurality of positions within the candidate peptide, if the candidate peptide is identified in step (b)(i), and (cii) substantially all permutations of the amino acid variations that occur within the known viral subtypes of the candidate peptide, above a given threshold frequency, if the candidate peptide is identified by step (b)(ii), and
(d) using bioinformatics to screen the antigens in the set of candidate antigens generated in step (c) against an MHC protein selected in step (a), to identify optimized antigens having a novel sequence, with respect to a corresponding know-subtype sequence, and a predicted binding affinity, with respect to the selected MHC protein, that is comparable or greater than that predicted for the natural wildtype sequence corresponding to that antigen.

2. The method of claim 1, wherein step (ci) is carried out by introducing at each amino-acid position within the candidate peptide, each of a set of amino acids that collectively, have properties representative of an entire set of natural amino acids.

3. The method of claim 1, wherein step (d) includes applying the predictive scoring MHC-binding algorithm to the sets of candidate peptides.

4. The method of claim 1, wherein step (d) is carried out to identify optimized viral antigens having a predicted binding affinity, with respect to the selected MHC protein, that is substantially greater than that predicted for the natural wildtype sequence of that peptide antigen.

5. The method of claim 1, which further includes repeating step (b)-(d) to identify a plurality of optimized viral antigens with different base sequences, and the vaccine or therapeutic composition produced contains two or more such optimized peptide antigens.

6. The method of claim 5, which further includes forming the vaccine or therapeutic composition by concatenating said two or more optimized viral antigens to form a multiple-antigen polypeptide, where the individual antigen peptides of the polypeptide are joined by a protease cleavable linkage.

7. The method of claim 6, wherein steps (b)-(d) are repeated for different viral-protein sequence regions, and the different sets of candidate antigens are screened in step (d) against the same MHC protein.

8. The method of claim 6, wherein steps (b)-(d) are repeated for different viral-protein sequence regions, and the different sets of candidate antigens are screened in step (d) against different MHC proteins.

9. The method of claim 1, for producing a vaccine or therapeutic composition against infection by an influenza A virus, wherein the viral protein-sequence region identified in step (a) is selected from the group consisting of SEQ IDS 1-15 and 18-33.

10. The method of claim 7, wherein the MHC protein against which the sets of candidate peptides are screened, and the optimized peptides identified by such screening are selected from the group consisting of:
(a) HLA B*5101, SEQ ID NOS: 16, 17; 34, 35
(b) HLA *0201, SEQ ID NOS: 91, 92
(c) HLA *0201, SEQ ID NOS: 93.
(d) HLA B44, SEQ ID NOS: 94.

11. The method of claim 1, for producing a vaccine or therapeutic composition against infection by a human immunodeficiency virus, wherein the viral protein-sequence region identified in step (a) is selected from the group consisting of SEQ IDS L-N.

12. A vaccine or therapeutic composition for use against infection by an influenza A virus, comprising a plurality of optimized antigens, in concatenated form, and selected from two of more from the group of optimized antigens selected from the group consisting of:
(a) SEQ ID NOS: 84-85;
(b) SEQ ID NOS: 86-88; and
(c) SEQ ID NOS: 89-90.


SEQUENCE LISTING (92 sequences)

SEQ ID NO: 1 - protein, 97 aa, Artificial Sequence
  Synthetic peptide antigen
MSLLTEVETPTRNGWECKCSDSSDPLVIAASIIGILHLILWILDRLFFKCIYRRLKYGLKRGPSTEGVPESMREEYRQEQQSAVDVDDGHFVSIELE

SEQ ID NO: 2 - protein, 97 aa, Artificial Sequence
  Synthetic peptide antigen
MSFLTEVETPIRNEWGCRCNGSSDPLTIAANIIGILHLTLWMLDRLFFKCIYRRFKYGLKGGPSTEGVPKSMREEYRKEQQSAVDTDDGHFVSIELE

SEQ ID NO: 3 - protein, 97 aa, Artificial Sequence
  Synthetic peptide antigen
MSLLTEVETPTKNGWECRCSDSSDPLVIAASIIGILHLILWILDRLFFKCIYRRLKYGLKRGPSTEGVPESMREEYRQEQQSAVDVDDGHFVNIELE

SEQ ID NO: 4 - protein, 97 aa, Artificial Sequence
  Synthetic peptide antigen
MSLLTEVETPTRNEWECRCSDSSDPLVVAASIIGILHLILWILDRLFFKCIYRRLKYGLKRGPSTAGVPESMREEYRQEQQSAVDVDDGHFVNIELE

SEQ ID NO: 5 - protein, 97 aa, Artificial Sequence; identical to SEQ ID NO: 4
  Synthetic peptide antigen

SEQ ID NO: 6 - protein, 97 aa, Artificial Sequence
  Synthetic peptide antigen
MSLLTEVETPTRNGWECKCSDSSDPLVIAASIIGILHLILWILDRLFFKCIYRRLKYGLKRGPSTEGVPESMREEYRQEQQSAVDVDDGHFVNIELE

SEQ ID NO: 7 - protein, 97 aa, Artificial Sequence
  Synthetic peptide antigen
MSLLTEVETPTRNGWECKCSDSSDPLVIAASIIGILHLILWILDRLFFKCIYRRLKYGLKRGPSTEGVPESMREEYRQEQQSAVDVDDSHFVNIELE

SEQ ID NO: 8 - protein, 97 aa, Artificial Sequence
  Synthetic peptide antigen
MSLLTEVETPTRNGWECKCSDSSDPLVVAASIIGILHLILWILDRLFFKCIYRRLKYGLKRGPSTEGVPESMREEYRQEQQSAVDVDDGHFVNIELE

SEQ ID NO: 9 - protein, 97 aa, Artificial Sequence
  Synthetic peptide antigen
MSLLTEVETPTRNGWGCRCSGSSDPLVVAASIXGILHLILWILDRLFFKCIYRRFKYGLKRGPSTEGVPESMREEYRQEQQSAVDVDDGHFVNIELE

SEQ ID NO: 10 - protein, 97 aa, Artificial Sequence
  Synthetic peptide antigen
MSLLTEVETPTRNGWECRCSDSSDPLVIAASIIGILHLILWILDRLFFKCIYRRLKYGLKRGPSTGGVPESMREEYRQEQQSAVDVDDGHFVNIELE

SEQ ID NO: 11 - protein, 97 aa, Artificial Sequence
  Synthetic peptide antigen
MSLLTEVETPTRNGWECRCSDSSDPLVIAASIIGILHLILWILDRLFFKCIYRRLKYGLKRGPSTEGVPESMREEYRQEQQSAVDVDDGHFVNIELE

SEQ ID NO: 12 - protein, 97 aa, Artificial Sequence
  Synthetic peptide antigen
MSLLTEVETPTRNGWECRCSDSSDPLVIAASIIGILHLILWILDRLFFKCIYRRLKYGLKRGPSTEGVPESMREEYRQEQQNAVDVDDGHFVNIELE

SEQ ID NO: 13 - protein, 97 aa, Artificial Sequence
  Synthetic peptide antigen
MSLLTEVETHTRSGWECRCSDSSDPLVIAASIIGILHLILWILDRLFFKCIYRRLKYGLKSGPSTEGVPESMREEYQQEKQSAVDVDDGHFVNIELE

SEQ ID NO: 14 - protein, 97 aa, Artificial Sequence
  Synthetic peptide antigen
MSLLTEVETPTRNEWECKCSDSSDPLVIAASIIGILHLTLWILDRLFFKCIYRRLKYGLKRGPSTEGVPESMREEYRQEQQNAVDVDDGHFVNIELE

SEQ ID NO: 15 - protein, 97 aa, Artificial Sequence; identical to SEQ ID NO: 6
  Synthetic peptide antigen

SEQ ID NO: 16 - protein, 9 aa, Artificial Sequence
  Synthetic peptide antigen
DPLXXAAXI

SEQ ID NO: 17 - protein, 15 aa, Artificial Sequence
  Synthetic peptide antigen
XXXWXCXCXXSSDPL

SEQ ID NO: 18 - protein, 229 aa, Artificial Sequence
  Synthetic peptide antigen
DSNTVSSFQVDCFLWHVRKRFADQELGDAPFLARLRRDQKSLRGRGSTLGLDIRTATHEGKHIVERILEEESDEALKMTIASVPAPRYLTDMTLEEMSRDWLMLIPKQKVTGSLCIRMDQAIVDKNITLKANFSVIFNRLEALILLRAFTDEGAIVGEISPLPSLPGHTDEDVKNAIGILIGGFEWNDNTVRVSETLQRFAWRSSDEDGRPPLSPKEKREMERTIEPEV

SEQ ID NO: 19 - protein, 225 aa, Artificial Sequence
  Synthetic peptide antigen
MDSNTVSSFQVDCFLWHVRKRFADQELGDAPFLDRLRRDQKSLRGRGNTLGLDIETATCAGKQIVERILEEESDEALKMPAPRYLTDMTLEEMSRDWFMLMPKQKVAGSLCIKMDQAIMDKNIILKANFSVIFDRLETLILLRAFTEEGAIVGEISPLPSLPGHTDEDVKNAIGVLIGGLEWNDNTVRVSETLQRFAWRSSDEDGRPPLPPNQKRKMARTIESEV

SEQ ID NO: 20 - protein, 222 aa, Artificial Sequence
  Synthetic peptide antigen
FQVDCFLWHVRKRFADQELGDAPFLDRLRRDQKSLRGRGSTLGLDIETATRAGKQIVERILEEESDEALKMTIASVPASRYLTDMTLEEMSRDWFMLMPKQKVAGSLCIRMDQAIMDKNIILKANFSVIFDRLETLILLRAFTEEGAIVGEISPLPSLPGHTDEDVKNAIGVLIGGLEWNDNTVRVSETLQRFAWRSSNEDGRPPLPPKQKRKMARTIESEV

SEQ ID NO: 21 - protein, 230 aa, Artificial Sequence
  Synthetic peptide antigen
MDSNTVSSFQVDCFLWHVRKRFADQELGDAPFLDRLRRDQKSLRGRGSTLGLDIDTATRAGKQIVERILEEESDEALKMTITSVPASRYLTDMTLEEMSRDWFMLMPKQKVASSLCIRMDQAIMDKNIILKANFSVIFDRLEALILLRAFTEEGAIVGEISPLPSLPGHTDEDVKNAIEVLIGGLEWNDNTVRVSETLQRFAWRSCNENGRPPLPPKQKRKMARTIESEV

SEQ ID NO: 22 - protein, 230 aa, Artificial Sequence
  Synthetic peptide antigen
MDSNTVSSFQVDCFLWHVRKRFADQELGDAPFLDRLRRDQKSLRGRGSTLGLDIETATHAGKQIVERILEEESDEALKMTIASVPASRYLTDMTLEEMSRDWFMLMPKQKVAGSLCIRMDQAIMDKNIILKANFSVIFDRLETLILLRAFTEEGAIVGEISPLPSLPGHTDEDVKNAIGVLIGGLEWNDNTVRVSETLQRFAWRSSNEDGRPPLPPKQKRKMARTIESEV

SEQ ID NO: 23 - protein, 230 aa, Artificial Sequence
  Synthetic peptide antigen
MDSNTVSSFQVDCFLWHVRKRFADQELGDAPFLDRLRRDQKSLRGRGSTLGLDIETATRAGKQIVERILEEESDEALKMTITSVPASRYLTDMTLEEMSRDWFMLMPKQKVAGSLCIRMDQAIMDKNIKLKANFSVIFDRLETLILLRAFTEEGAIVGEISPLPSLPGHTDEDVKNAIGVLIGGLEWNDNTVRVSETLQRFAWRSSNEDGRPPLPPKQKRKMARTIESEV

SEQ ID NO: 24 - protein, 230 aa, Artificial Sequence
  Synthetic peptide antigen
MDSNTVSSFQVDCFLWHVRKRFADQELGDAPFLDRLRRDQKSLRGRGSTLGLDIETATRAGKQIVERILEEESDEALKMTIASVPTSRYLTDMTLEEMSRDWFMLMPKQKVAGSLCIRMDQAIMDKNIILKANFSVIFDRLETLILLRAFTEEGAIVGEISPLPSLPGHTDEDVKNAIGVLIGGLEWNDNTVRVSETLQRFAWRSSNEDGRPPLPPKQKRKMARTIESEV

SEQ ID NO: 25 - protein, 230 aa, Artificial Sequence
  Synthetic peptide antigen
MDSNTVSSFQVDCFLWHVRKRFADQELGDAPFLDRLRRDQKSLRGRGSTLGLDIETATRAGKQIVERILEEESDEALKMTIASVPASRYLTDMTLEEMSRDWFMLMPKQKVAGSLCIRMDQAIMDKNIILKANFSVIFDRLETLILLRAFTEEGAIVGEISPLPSLPGHTDEDVKNAIGVLIGGFEWNDNTVRVSETLQRFAWRSSNENGRPPLPPKQKRKMARTIESEV

SEQ ID NO: 26 - protein, 230 aa, Artificial Sequence
  Synthetic peptide antigen
MDSNTVSGFQVDCFLWHVRKRFADQELGDAPFLDRLRRDQKSLRGRGSTLGLDIETATRAGKQIVERILEEESDEALQMTIASVPASRYLTDMTLEEMSRDWFMLMPKQKVAGSLCIRMDQAIMDKNIILKANFSVIFDRLETLILLRAFTEEGAIVGEISPLPSLPGHTDEDVKNAIGVLIGGLEWNDNTVRVSETLQRFAWRSSNEDGRPPLPPKQKRKMARTIESEV

SEQ ID NO: 27 - protein, 229 aa, Artificial Sequence
  Synthetic peptide antigen
MDSNTITSFQVDCYLWHIRKLLSMRDMCDAPFDDRLRRDQKALKGRGSTLGLDLRVATIEGKKIVEDILKSEPDEILKIAIASIPAPRYITDMSMEEISREWYMLMPRQKITGGLVVKMDQAIMDKRIILKANFSVLFNQLETLVSLRAFTDDGSIVAEISPFPSMPGHSAEDVKNAIGILIGGLEWNDNSVRASENIQRFAWGVCDENRGPSLPPNQKCYMAGRVESK

SEQ ID NO: 28 - protein, 230 aa, Artificial Sequence
  Synthetic peptide antigen
MDSNTVSSFQVDCFLWHVRKRFADQELGDAPFLDRLRRDQKSLRGRGSTLGLDIETATRAGKQIVERILEEESDEALKMTIASVPASRYLTDMTLEEMSRDWFMLMPKQKVAGSLCIRMDQAIMDKNITLKANFSVIFDRLETLILLRAFTDEGAIVGEISPLPSLPGHTDEDVKNAIGVLIGGLEWNDNTVRVSETLQRFAWRSSNEDGRPPLPPKQKRKMARTVESEV

SEQ ID NO: 29 - protein, 230 aa, Artificial Sequence
  Synthetic peptide antigen
MDSNTVSSFQVDCFLWHVRKRFADQGLGDAPFLDRLRRDQKSLRGRGSTLGLDIETATRAGKQIVERILEEESDEALKMTIASVPASRYLTDMTLEEMSRDWFMLMPKQKVAGSLCIRMDQAIMDKNIILKANFSVIFDRLETLILLRAFTEEGAIVGEISPLPSLPGHTDEDVKNAIGVLIGGLEWNDNTVRVSETLQRFAWRGSNEDGRPPLPPKQKRKMARTIESEV

SEQ ID NO: 30 - protein, 230 aa, Artificial Sequence
  Synthetic peptide antigen
MDSNTVSSFQVDCFLWHVRKRFADQDMGDAPFLDRIRRDQKSLKGRSITIGMDIEAATRAGKQIIERILDEESDKALKMNIASVPAPRYVTDMTPEEMSRDWFMLMPKQKFAGPLCIRMDQAILDKNIILKANFSVVFDRLETLILLRAFTSEGAIVGEISQLPSLPGHTNEDVKNAIGILIGGLEWNDNTVRVSETLQRFAWGSSNENGRPPFAPKQERKMAGTVESEV

SEQ ID NO: 31 - protein, 230 aa, Artificial Sequence
  Synthetic peptide antigen
MDPNTVSSFQVDCFLWHVRKRVADQELGDAPFLDRLRRDQKSLRGRGSTLGLDIKTATRAGKQIVERILKEESDEALKMTMASVPASRYLTDMTLEEMSRDWSMLIPKQKVAGPLCIRMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

SEQ ID NO: 32 - protein, 230 aa, Artificial Sequence
  Synthetic peptide antigen
MDSHTVSSFQVDCFLWHVRKQVADKDLGDAPFLDRLRRDQKSLKGRGSTLGLNIETATCVGKQIVERILKEESDEAFKMTMASALASRYLTDMTVEEMSRDWFMLMPKQKVAGPLCVRMDQAIMDKNIILKANFSVIFDRLENLTLLRAFTEEGAIVGEISPLPSFPGHTNEDVKNAIGVLIGGLEWNDNTVRVSETLQRFAWRSSNETGGPPFTTTQKRKMAGTIRSEV

SEQ ID NO: 33 - protein, 230 aa, Artificial Sequence
  Synthetic peptide antigen
MDSNTVSSFQVDCFLWHVRKRFADQELGDAPFLDRLRRDQKSLRGRGSTLGLDIETATRAGKQIVERILEEESDEALKMTIASVPASRYLTDMTLEEMSRDWFMLMPKQKVAGSLCIRMDQAIMDKNIILKANFSVIFGRLETLILLRAFTEEGAIVGEISPLPSLPGHTDEDVKNAIGVLIGGLEWNDNTVRVSETLQRFAWRSSNEDGRPPLPPKQKRKMARTIESEV

SEQ ID NO: 34 - protein, 9 aa, Artificial Sequence
  Synthetic peptide antigen
IFXRLEXLX

SEQ ID NO: 35 - protein, 14 aa, Artificial Sequence
  Synthetic peptide antigen
DXXXKXXXXSXXAX

SEQ ID NO: 36 - protein, 9 aa, Artificial Sequence
  Synthetic peptide antigen
RPLVIAASI

SEQ ID NO: 37 - protein, 9 aa, Artificial Sequence
  Synthetic peptide antigen
RPLVIAASI

SEQ ID NO: 38 - protein, 9 aa, Artificial Sequence
  Synthetic peptide antigen
RPLVIAASI

SEQ ID NO: 39 - protein, 9 aa, Artificial Sequence
  Synthetic peptide antigen
DPLRIAASI

SEQ ID NO: 40 - protein, 9 aa, Artificial Sequence
  Synthetic peptide antigen
DPLVRAASI

SEQ ID NO: 41 - protein, 9 aa, Artificial Sequence
  Synthetic peptide antigen
DPLVIRASI

SEQ ID NO: 42 - protein, 9 aa, Artificial Sequence
  Synthetic peptide antigen
DPLVIARSI

SEQ ID NO: 43 - protein, 9 aa, Artificial Sequence
  Synthetic peptide antigen
DPLVIAARI

SEQ ID NO: 44 - protein, 9 aa, Artificial Sequence
  Synthetic peptide antigen
DPLVIAASR

SEQ ID NO: 45 - DNA, 27 bp, Artificial Sequence
  DNA coding sequence for synthetic peptide antigen
cgcccgctggtgatcgccgccagcatc

SEQ ID NO: 46 - DNA, 27 bp, Artificial Sequence
  DNA coding sequence for synthetic peptide antigen
gaccgcctggtgatcgccgccagcatc

SEQ ID NO: 47 - DNA, 27 bp, Artificial Sequence
  DNA coding sequence for synthetic peptide antigen
gacccgcgcgtgatcgccgccagcatc

SEQ ID NO: 48 - DNA, 27 bp, Artificial Sequence
  DNA coding sequence for synthetic peptide antigen
gacccgctgcgcatcgccgccagcatc

SEQ ID NO: 49 - DNA, 27 bp, Artificial Sequence
  DNA coding sequence for synthetic peptide antigen
gacccgctggtgcgcgccgccagcatc

SEQ ID NO: 50 - DNA, 27 bp, Artificial Sequence
  DNA coding sequence for synthetic peptide antigen
gacccgctggtgatccgcgccagcatc

SEQ ID NO: 51 - DNA, 27 bp, Artificial Sequence
  DNA coding sequence for synthetic peptide antigen
gacccgctggtgatcgcccgcagcatc

SEQ ID NO: 52 - DNA, 27 bp, Artificial Sequence
  DNA coding sequence for synthetic peptide antigen
gacccgctggtgatcgccgcccgcatc

SEQ ID NO: 53 - DNA, 27 bp, Artificial Sequence
  DNA coding sequence for synthetic peptide antigen
gacccgctggtgatcgccgccagccgc

SEQ ID NO: 54 - protein, 9 aa, Artificial Sequence
  Synthetic peptide antigen
DPLVIAASR

SEQ ID NO: 55 - protein, 9 aa, Artificial Sequence
  Synthetic peptide antigen
DPLVSAASI

SEQ ID NO: 56 - protein, 9 aa, Artificial Sequence
  Synthetic peptide antigen
DPLVIAASY

SEQ ID NO: 57 - protein, 9 aa, Artificial Sequence
  Synthetic peptide antigen
DPLVIAASY

SEQ ID NO: 58 - protein, 9 aa, Artificial Sequence
  Synthetic peptide antigen
DPLTIAASI

SEQ ID NO: 59 - protein, 9 aa, Artificial Sequence
  Synthetic peptide antigen
DPLVVAASI

SEQ ID NO: 60 - protein, 9 aa, Artificial Sequence
  Synthetic peptide antigen
DPLTVAASI

SEQ ID NO: 61 - protein, 9 aa, Artificial Sequence
  Synthetic peptide antigen
DPLTIAANI

SEQ ID NO: 62 - protein, 9 aa, Artificial Sequence
  Synthetic peptide antigen
DPLVVAANI

SEQ ID NO: 63 - protein, 9 aa, Artificial Sequence
  Synthetic peptide antigen
DPLTVAANI

SEQ ID NO: 64 - DNA, 90 bp, Artificial Sequence
  DNA coding sequence for synthetic peptide antigen
gccggatccaccatgggtcgtaaagcaatcatggagaaaaatatcatgctgaagcgtgcaattatggagaagaacatcattctgaagaaa

SEQ ID NO: 65 - DNA, 66 bp, Artificial Sequence
  DNA coding sequence for synthetic peptide antigen
gctatcatggaaaagactatcatcctgcgtaaggcaattaccgagaaatcgatcattctgaaacgt

SEQ ID NO: 66 - DNA, 66 bp, Artificial Sequence
  DNA coding sequence for synthetic peptide antigen
gctatcatggacaagaacatcatgttgaaaaaggccattatggataagaacatcattctgcgtaaa

SEQ ID NO: 67 - DNA, 66 bp, Artificial Sequence
  DNA coding sequence for synthetic peptide antigen
gcaattaccgagaagactattattttgaaacgtgccattaccgaaaaatctatcatcctgaaaaag

SEQ ID NO: 68 - DNA, 66 bp, Artificial Sequence
  DNA coding sequence for synthetic peptide antigen
ggtatcctgggtttcgtgtttaccttgcgtaaagtcgagaccccgattcgtaacgaatggaagcgt

SEQ ID NO: 69 - DNA, 66 bp, Artificial Sequence
  DNA coding sequence for synthetic peptide antigen
gtggagactcctactcgtaacgaatggaagaaaggcctgttgggcttcgtgttcactctgcgtaag

SEQ ID NO: 70 - DNA, 43 bp, Artificial Sequence
  DNA coding sequence for synthetic peptide antigen
ggtatcttgggcttcgtcttcactgtgaaacgtctcgagccgg

SEQ ID NO: 71 - DNA, 84 bp, Artificial Sequence
  DNA coding sequence for synthetic peptide antigen
cagaatgatgttcttctccataattgcacgcttcagcatgatatttttctccatgattgctttacgacccatggtggatccggc

SEQ ID NO: 72 - DNA, 66 bp, Artificial Sequence
  DNA coding sequence for synthetic peptide antigen
cagaatgatcgatttctcggtaattgccttacgcaggatgatagtcttttccatgatagctttctt

SEQ ID NO: 73 - DNA, 66 bp, Artificial Sequence
  DNA coding sequence for synthetic peptide antigen
cagaatgatgttcttatccataatggcctttttcaacatgatgttcttgtccatgatagcacgttt

SEQ ID NO: 74 - DNA, 66 bp, Artficial Sequence
  DNA coding sequence for synthetic peptide antigen
caggatgatagatttttcggtaatggcacgtttcaaaataatagtcttctcggtaattgctttacg

SEQ ID NO: 75 - DNA, 66 bp, Artificial Sequence
  DNA coding sequence for synthetic peptide antigen
ccattcgttacgaatcggggtctcgactttacgcaaggtaaacacgaaacccaggatacccttttt

SEQ ID NO: 76 - DNA, 66 bp, Artificial Sequence
  DNA coding sequence for synthetic peptide antigen
cagagtgaacacgaagcccaacaggcctttcttccattcgttacgagtaggagtctccacacgctt

SEQ ID NO: 77 - DNA, 49 bp, Artificial Sequence
  DNA coding sequence for synthetic peptide antigen
ccggctcgagacgtttcacagtgaagacgaagcccaagatacccttacg

SEQ ID NO: 78 - protein, 9 aa, Artificial Sequence
  Synthetic peptide antigen
AIMEKNIML

SEQ ID NO: 79 - protein, 9 aa, Artificial Sequence
  Synthetic peptide antigen
AIMEKNIIL

SEQ ID NO: 80 - protein, 9 aa, Artificial Sequence
  Synthetic peptide antigen
AIMEKTIIL

SEQ ID NO: 81 - protein, 9 aa, Artificial Sequence
  Synthetic peptide antigen
AITEKSIIL

SEQ ID NO: 82 - protein, 9 aa, Artificial Sequence
  Synthetic peptide antigen
AIMDKNIML

SEQ ID NO: 83 - protein, 9 aa, Artificial Sequence
  Synthetic peptide antigen
AIMDKNIIL

SEQ ID NO: 84 - protein, 9 aa, Artificial Sequence
  Synthetic peptide antigen
AITEKTIIL

SEQ ID NO: 85 - protein, 9 aa, Artificial Sequence
  Synthetic peptide antigen
AIMEKSIIL

SEQ ID NO: 86 - protein, 9 aa, Artificial Sequence
  Synthetic peptide antigen
GILGFVFTL

SEQ ID NO: 87 - protein, 9 aa, Artificial Sequence
  Synthetic peptide antigen
GLLGFVFTL

SEQ ID NO: 88 - protein, 9 aa, Artificial Sequence
  Synthetic peptide antigen
GLLGFVFTV

SEQ ID NO: 89 - protein, 9 aa, Artificial Sequence
  Synthetic peptide antigen
VETPIRNEW

SEQ ID NO: 90 - protein, 9 aa, Artificial Sequence
  Synthetic peptide antigen
VETPTRNEW

SEQ ID NO: 91 - protein, 9 aa, Artificial Sequence
  Synthetic peptide antigen
IFXRLEXLI

SEQ ID NO: 92 - protein, 19 aa, Artificial Sequence
  Synthetic peptide antigen
DXXXXKXASXXXXSXXAXR